(19) United States
(12) Patent Application Publication
(10) Pub. No.: US 2026/0102101 A1
(43) Pub. Date: Apr. 16, 2026

(54) HEART SOUND CONFIRMATION OF ATRIAL ARRHYTHMIA

(71) Applicant: Cardiac Pacemakers, Inc., St. Paul, MN (US)

(72) Inventors: Viktoria A. AVERINA, Shoreview, MN (US); David L. PERSCHBACHER, Blaine, MN (US); Pramodsingh Hirasingh THAKUR, Woodbury, MN (US); Jonathan Bennett SHUTE, Eagan, MN (US); Mojgan GOFTARI, Shoreview, MN (US)

(21) Appl. No.: 19/354,543

(22) Filed: Oct. 9, 2025

Related U.S. Application Data
(60) Provisional application No. 63/705,931, filed on Oct. 10, 2024.

(51) Int. Cl. A61B 5/364; A61B 5/352; A61B 7/02

(52) U.S. Cl. CPC A61B 5/364; A61B 5/352; A61B 7/02

(57) ABSTRACT

Systems and methods are disclosed for improving arrhythmia detection to optimize resources of a medical device system, including detecting an arrhythmia episode using cardiac electrical information and analyzing cardiac mechanical information occurring over a final portion of a cardiac cycle of the detected arrhythmia episode to confirm the detected arrhythmia episode or to determine a type of the detected arrhythmia episode. The systems and methods can provide a control signal to control a mode or operation of a component of the medical device system based on the confirmation of the detected arrhythmia episode or the determined type of the detected arrhythmia episode to optimize resources of the medical device system.

### CROSS-REFERENCE TO RELATED APPLICATION(S)

This application claims the benefit of and priority to U.S. Provisional Application No. 63/705,931 filed Oct. 10, 2024, the entire contents of which is hereby incorporated by reference.

### TECHNICAL FIELD

This document relates generally to medical devices and more particularly to heart sound confirmation of atrial arrhythmia detection.

### BACKGROUND

Ambulatory medical devices (AMDs), including implantable, subcutaneous, wearable, insertable, or one or more other medical devices, etc., can monitor, detect, or treat various conditions, including heart failure (HF), arrhythmia, etc. Ambulatory medical devices can include sensors to sense physiologic information from a patient and one or more circuits to detect one or more physiologic events using the sensed physiologic information or transmit sensed physiologic information or detected physiologic events to one or more remote devices. Additionally, ambulatory medical devices can be configured to provide electrical stimulation or one or more other therapies or treatments to the patient, such as to improve cardiac function, etc.

Ambulatory patient monitoring can provide early detection of worsening patient condition, including worsening heart failure, arrhythmia, etc. Accurate identification of patients or groups of patients at an elevated risk of future adverse events may control mode or feature selection or resource management of one or more medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of medical devices, and reduce healthcare costs. In addition, in situations where different operating modes, features, or therapies are available, correctly monitoring, detecting, and identifying patient status, including improving or worsening patient condition, and modifying one or more medical device functions based thereon, can improve medical device efficiency, such as by reducing unnecessary resource consumption, thereby extending the usable life of the ambulatory medical device.

### SUMMARY

Systems and methods are disclosed for improving arrhythmia detection to optimize resources of a medical device system, including detecting an arrhythmia episode using cardiac electrical information and analyzing cardiac mechanical information occurring over a final portion of a cardiac cycle of the detected arrhythmia episode to confirm the detected arrhythmia episode or to determine a type of the detected arrhythmia episode. The systems and methods can provide a control signal to control a mode or operation of a component of the medical device system based on the confirmation of the detected arrhythmia episode or the determined type of the detected arrhythmia episode to optimize resources of the medical device system.

An example of subject matter (e.g., a medical device system for improving arrhythmia detection to optimize resources of the medical device system) may comprise means for receiving physiologic information of a patient, the physiologic information including cardiac electrical and cardiac mechanical information, means for detecting an arrhythmia episode using the cardiac electrical information, means for analyzing the cardiac mechanical information occurring over a final portion of a cardiac cycle of the detected arrhythmia episode to confirm the detected arrhythmia episode or to determine a type of the detected arrhythmia episode based on analysis of the cardiac mechanical information, and means for providing a control signal to control a mode or operation of the medical device system based on the confirmation of the detected arrhythmia episode or the determined type of the detected arrhythmia episode to optimize resources of the medical device system.

In an example, which may be combined with any one or more examples described herein, the means for receiving physiologic information of the patient comprises a signal receiver circuit configured to receive the physiologic information of the patient including cardiac electrical and cardiac mechanical information, and the means for receiving, detecting, analyzing, and providing comprises an assessment circuit configured to detect the arrhythmia episode using the cardiac electrical information, analyze the cardiac mechanical information occurring over the final portion of the cardiac cycle of the detected arrhythmia episode to confirm the detected arrhythmia episode or to determine the type of the detected arrhythmia episode based on analysis of the cardiac mechanical information, and provide the control signal to control the mode or operation of the medical device system based on the confirmation of the detected arrhythmia episode or the determined type of the detected arrhythmia episode to optimize resources of the medical device system.

An example of subject matter (e.g., a medical device system for improving arrhythmia detection to optimize resources of the medical device system) may comprise a signal receiver circuit configured to receive physiologic information of a patient, the physiologic information including cardiac electrical and cardiac mechanical information, and an assessment circuit configured to detect an arrhythmia episode using the cardiac electrical information, analyze the cardiac mechanical information occurring over a final portion of a cardiac cycle of the detected arrhythmia episode to confirm the detected arrhythmia episode or to determine a type of the detected arrhythmia episode based on analysis of the cardiac mechanical information, and provide a control signal to control a mode or operation of the medical device system based on the confirmation of the detected arrhythmia episode or the determined type of the detected arrhythmia episode to optimize resources of the medical device system.

In an example, which may be combined with any one or more examples described herein, the assessment circuit is configured to trigger sensing of or receiving the cardiac mechanical information, including heart sound information, based on the detected arrhythmia episode.

In an example, which may be combined with any one or more examples described herein, the cardiac electrical information includes heart rate information, wherein to analyze the cardiac mechanical information includes to confirm the detected arrhythmia episode and to determine the type of the detected arrhythmia episode based on the analysis of the cardiac mechanical information, wherein to determine the type of arrhythmia episode includes to determine one of a plurality of types of arrhythmia episode including an atrial tachycardia episode, an atrial fibrillation episode, or an atrial flutter episode.

In an example, which may be combined with any one or more examples described herein, the assessment circuit is configured to trigger sampling and storage of an electrocardiogram signal at a sampling rate and storage size greater than the heart rate information in response to the cardiac mechanical information exceeding a threshold.

In an example, which may be combined with any one or more examples described herein, the cardiac mechanical information includes an S4 heart sound value, wherein the assessment circuit is configured to determine and analyze the S4 heart sound value occurring over an S4 window of a first cardiac cycle and ending at a beginning of a second cardiac cycle immediately following the first cardiac cycle at a subsequent R wave or S1 heart sound.

In an example, which may be combined with any one or more examples described herein, the assessment circuit is configured to detect a change in the S4 heart sound value for the detected arrhythmia episode with respect to a baseline value and to confirm the detected arrhythmia episode and to determine the type of the detected arrhythmia episode based on the detected change.

In an example, which may be combined with any one or more examples described herein, the cardiac mechanical information includes a pre-S1 heart sound or pre-R-wave value, wherein the assessment circuit is configured to determine and analyze the pre-S1 heart sound or the pre-R-wave value occurring over a respective pre-S1 heart sound or pre-R-wave period of time having a pre-determined duration occurring over a final portion of a first cardiac cycle and ending at a beginning of a second cardiac cycle immediately following the first cardiac cycle at a subsequent R wave or S1 heart sound, wherein the pre-determined duration is between 100 ms and 250 ms, wherein the assessment circuit is configured to confirm the detected arrhythmia episode and to determine the type of the detected arrhythmia episode based on the pre-S1 heart sound or the pre-R-wave value.

In an example, which may be combined with any one or more examples described herein, the cardiac mechanical information includes a post-S2 heart sound value, wherein the assessment circuit is configured to determine and analyze the post-S2 heart sound value occurring over a post-S2 heart sound time period beginning at one of an end of an S2 heart sound or a beginning or an end of an S3 heart sound or an S3 heart sound window of a first cardiac cycle and ending at a beginning of a second cardiac cycle immediately following the first cardiac cycle at a subsequent R wave or S1 heart sound, wherein the assessment circuit is configured to confirm the detected arrhythmia episode and to determine the type of the detected arrhythmia episode based on the post-S2 heart sound value.

In an example, which may be combined with any one or more examples described herein, the cardiac mechanical information includes a diastolic interval, wherein the assessment circuit is configured to determine and analyze the diastolic interval as a time period beginning at a beginning of an S2 heart sound of a first cardiac cycle and ending at a beginning of a second cardiac cycle immediately following the first cardiac cycle at a subsequent R wave or S1 heart sound, wherein the assessment circuit is configured to confirm the detected arrhythmia episode and to determine the type of the detected arrhythmia episode based on the diastolic interval.

In an example, which may be combined with any one or more examples described herein, the assessment circuit is configured to detect a reduction in the diastolic interval for the detected arrhythmia episode with respect to a baseline value and to determine the type of the detected arrhythmia episode based on the detected reduction exceeding a threshold.

In an example, which may be combined with any one or more examples described herein, the assessment circuit is configured to determine at least one of an S4 heart sound value, a pre-S1 heart sound or pre-R-wave value occurring over a pre-S1 heart sound or pre-R-wave period of time having a pre-determined duration of 100 ms occurring over a final portion of the first cardiac cycle and ending at the beginning of the second cardiac cycle, or a post-S2 heart sound value occurring over a post-S2 heart sound time period beginning at one of an end of the S2 heart sound or a beginning or an end of an S3 heart sound or an S3 heart sound window of the first cardiac cycle and ending at the beginning of the second cardiac cycle determine a composite cardiac mechanical measure as a function of the determined diastolic interval and the determined at least one of the S4 heart sound value, the pre-S1 heart sound or pre-R-wave value, or the post-S2 heart sound value and determine the type of the detected arrhythmia episode based on the determined composite cardiac mechanical measure.

In an example, which may be combined with any one or more examples described herein, the subject matter may include a heart sound sensor configured to sense the cardiac mechanical information from the patient and a cardiac electrical sensor configured to sense the cardiac electrical information from the patient, wherein the signal receiver circuit is configured to receive the sensed cardiac mechanical information from the heart sound sensor and the cardiac electrical information from the cardiac electrical sensor.

In an example, which may be combined with any one or more examples described herein, to control the mode or operation of the medical device system includes to control sensing, storage, or transmission of the cardiac electrical information or the cardiac mechanical information or display of detected arrhythmia episodes to optimize resources of the medical device system.

In an example, which may be combined with any one or more examples described herein, the assessment circuit is configured to perform frequency analysis on an ensemble average of cardiac mechanical information over the detected arrhythmia episode to determine a value of frequency density of the ensemble average of cardiac mechanical information, the ensemble average representative of an aggregate of cardiac mechanical information over multiple cardiac cycles of the detected arrhythmia episode and determine the type of the detected arrhythmia episode based on the determined value of frequency density of the ensemble average of cardiac mechanical information.

An example of subject matter (e.g., a method for improving arrhythmia detection by a medical device system to optimize resources of the medical device system) may comprise receiving physiologic information of a patient using a signal receiver circuit, the physiologic information including cardiac electrical and cardiac mechanical information, and, using an assessment circuit, detecting an arrhythmia episode using the cardiac electrical information, analyzing the cardiac mechanical information occurring over a final portion of a cardiac cycle of the detected arrhythmia episode to confirm the detected arrhythmia episode or to determine a type of the detected arrhythmia episode based on analysis of the cardiac mechanical information, and providing a control signal to control a mode or operation of the medical device system based on the confirmation of the detected arrhythmia episode or the determined type of the detected arrhythmia episode to optimize resources of the medical device system.

In an example, which may be combined with any one or more examples described herein, the subject matter comprises triggering sensing of or receiving the cardiac mechanical information, including heart sound information, based on the detected arrhythmia episode.

In an example, which may be combined with any one or more examples described herein, receiving the cardiac electrical information includes receiving heart rate information, wherein analyzing the cardiac mechanical information includes confirming the detected arrhythmia episode and to determine the type of the detected arrhythmia episode based on the analysis of the cardiac mechanical information, wherein determining the type of arrhythmia episode includes determining one of a plurality of types of arrhythmia episode including an atrial tachycardia episode, an atrial fibrillation episode, or an atrial flutter episode.

In an example, which may be combined with any one or more examples described herein, the subject matter comprises triggering sampling and storing an electrocardiogram signal at a sampling rate and storage size greater than the heart rate information in response to the cardiac mechanical information exceeding a threshold.

In an example, which may be combined with any one or more examples described herein, receiving the cardiac mechanical information includes receiving an S4 heart sound value, wherein analyzing the cardiac mechanical information includes determining and analyzing the S4 heart sound value occurring over an S4 window of a first cardiac cycle and ending at a beginning of a second cardiac cycle immediately following the first cardiac cycle at a subsequent R wave or S1 heart sound.

In an example, which may be combined with any one or more examples described herein, analyzing the S4 heart sound value comprises detecting a change in the S4 heart sound value for the detected arrhythmia episode with respect to a baseline value and confirming the detected arrhythmia episode and determining the type of the detected arrhythmia episode based on the detected change.

In an example, a system, method, or apparatus may optionally combine any portion or combination of any portion of any one or more of the examples described herein, may optionally combine any portion or combination of any portion of any one or more of the examples described herein to comprise “means for” performing any portion of any one or more of the functions, operations, or methods of the examples described herein, or at least one “non-transitory machine-readable medium” including instructions that, when performed by a machine, cause the machine to perform any portion of any one or more of the functions or methods of the examples described herein.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example relationship between physiologic parameters over time.

FIGS. 2-3 illustrates example methods for improving arrhythmia detection.

FIG. 4 illustrates an example medical device system.

FIG. 5 illustrates an example patient management system.

FIG. 6 illustrates an example implantable medical device system.

FIG. 7 illustrates example implantable medical devices.

FIG. 8 illustrates an example remote patient management system.

FIG. 9 illustrates an example machine upon which any one or more of the techniques discussed herein may perform.

### DETAILED DESCRIPTION

Ambulatory medical devices are devices configured to be implanted in or otherwise positioned on or about patients to monitor physiologic information, such as cardiac electrical, heart sound, respiration, impedance, pressure, physical activity, or other physiologic information or one or more other physiologic parameters of the patient, or to provide electrical stimulation or one or more other therapies or treatments to optimize or control one or more body functions of the patient, such as contractions of a heart, etc. Ambulatory medical devices can include implantable or external (e.g., wearable) devices configured to monitor or provide stimulation to the patient.

Ambulatory medical devices can provide different monitoring, storage, communication, or therapy using different modes, however, with different power and resource requirements and varying effectiveness for different patients. For example, a variety of therapy modalities are available to patients, but not all patients receive the optimal medical device, therapy mode, or therapy parameter settings at first programming. One common reason for suboptimal ambulatory medical device programming is detection or determination of different events, conditions, or indications. Optimal programming depends on, among other things, accurate detection and determination of different events, conditions, or indications. Desired clinical outcomes can include, among others, cardiac capture, selection of pacing or sensing vectors, pacing mode, resource usage associated with communication or transmission of physiologic data from the ambulatory medical device or storage of physiologic data, etc.

The present inventors have recognized, among other things, systems and methods to improve detection or confirmation of an arrhythmia episodes or differentiation between different types of arrhythmia episodes using one or more of cardiac electrical information (e.g., electrocardiogram (ECG) information, heart rate information, etc.), cardiac mechanical information (e.g., heart sound information, diastolic interval information, etc.), or a combination thereof. Three common types of atrial arrhythmias (AA) are atrial fibrillation (AF), atrial tachycardia (AT), and atrial flutter (AFL). Distinguishing between different types of arrhythmia episodes represents a significant clinical challenge, particularly in certain patient populations.

Atrial fibrillation is characterized by chaotic and irregular electrical activity in the atria, resulting in rapid and disorganized atrial contractions, which leads to an irregular ventricular response and often presents as random heart rate patterns in cardiac electrical information (e.g., an electrocardiogram). Atrial flutter and atrial tachycardia represent distinct types of atrial arrhythmias, each characterized by rapid and often abnormal contractions of the atria. Unlike atrial fibrillation, where atrial activity is chaotic and irregular, both atrial flutter and atrial tachycardia exhibit organized but abnormally fast rhythms. In atrial flutter, the rapid atrial rhythm is typically caused by reentrant electrical propagation in the atria, where electrical signals propagate in a circular path. This results in the atria contracting at a rate that is significantly faster than normal sinus rhythm, often leading to a characteristic sawtooth pattern on electrocardiogram recordings.

Atrial tachycardia, while also presenting with a rapid atrial rate, is typically caused by an ectopic focus in the atria that fires rapidly, rather than a reentrant circuit. This results in a regular, fast atrial rhythm that can be difficult to distinguish from atrial flutter or even normal sinus tachycardia on an electrocardiogram. Both atrial flutter and atrial tachycardia can present with regular ventricular responses, making them challenging to differentiate from each other and from normal sinus rhythm, particularly when the flutter waves or P waves are obscured by fast QRS complexes.

The regular nature of both atrial flutter and atrial tachycardia underscores the importance of advanced detection that utilizes and incorporates both cardiac electrical (e.g., electrocardiogram) and cardiac mechanical (e.g., heart sound) analysis. Such incorporation can improve accurate identification and differentiation of arrhythmias from each other, from atrial fibrillation, or from normal sinus rhythm. By analyzing heart sounds in addition to electrocardiogram information, the systems and methods described herein can provide a more comprehensive approach to arrhythmia detection, potentially improving the accuracy of diagnosis and subsequent management of these clinically significant atrial arrhythmias and optimizing the use and resources of ambulatory medical devices.

A sizable subset of patients with atrial fibrillation have or are at risk of other atrial arrhythmias. Patients with structural heart disease, those who have undergone post-cardiac surgery, those undergoing cancer treatments, or combinations thereof, are at increased risk for atrial arrhythmias, not limited to atrial fibrillation, and associated complications. The systems and methods described herein are particularly relevant for patients with implantable cardiac monitors (ICM), transvenous devices (TVD), subcutaneous implantable cardioverter-defibrillators (SICD), wearable monitors, or other ambulatory medical devices.

Arrhythmia episodes, including potential arrhythmia episodes, such as atrial fibrillation, atrial tachycardia, or atrial flutter episodes or potential episodes, can be detected using sensed or received cardiac electrical information, including, for example, detected atrial or ventricular events (e.g., heart beats, R waves, P waves, etc.) or intervals therebetween occurring within a detection window, often between 30 seconds and 2 minutes, though in certain examples longer or shorter. Ambulatory medical devices can determine, for example, using timing information between events, in certain examples, in combination with one or more other detected events, whether an arrhythmia is present or not in each detection window, and can additionally determine to store or transmit sensed or detected information, such as for transmission to a remote device, based on the determination. In certain examples, the ambulatory medical device can aggregate information from multiple sensors, detect various events using information from each sensor separately or in combination, update a detection status based on the information, and transmit a message or an alert to one or more remote devices that a detection has been made, that information has been stored or transmitted, such that one or more additional processes or systems can use the stored or transmitted detection or information for one or more other review or processes.

Example arrhythmia detection algorithms are described, for example, in the commonly assigned Krueger et al. U.S. patent application Ser. No. 14/825,669, titled “Atrial Fibrillation Detection Using Ventricular Rate Variability” (herein, “the '669 application”); Perschbacher et al. U.S. patent application Ser. No. 15/082,440, titled “Atrial Fibrillation Detection” (herein, “the '440 application”); Krueger et al. U.S. patent application Ser. No. 15/341,565, titled “Method and Apparatus for Enhancing Ventricular Based Atrial Fibrillation Detection Using Atrial Activity” (herein, “the '565 application”); and Perschbacher et al. U.S. patent application Ser. No. 15/864,953, titled “Atrial Fibrillation Discrimination Using Heart Rate Clustering” (herein, “the '953 application”), each of which are hereby incorporated by reference in their entireties, including their disclosure of atrial fibrillation detection and atrial fibrillation detection algorithms including, for example: atrial fibrillation detection using pairs of ventricular information detected from a ventricle, including rate changes and rate change characteristics, and determination of valid heart beats or intervals using various characteristics, including threshold rates, intervals, morphology criterion, etc., such as disclosed in the '669 application; atrial fibrillation detection using a distribution of ventricular depolarization intervals, such as disclosed in the '440 application; atrial fibrillation detection using atrial activity scores from an atrial detection window prior to a detected ventricular polarization, such as disclosed in the '565 application; atrial fibrillation discrimination using clustered depolarization information, such as disclosed in the '953 application, etc.

Existing systems and methods to detect arrhythmias commonly rely on electrocardiogram-based algorithms that often require adjudication by a clinician. The quality of adjudication varies with clinician skill and can result in under-diagnosis and under-treatment of non-fibrillation atrial arrhythmias, increasing risks of mortality, hospital admissions for arrhythmia-related complications, stroke, and heart failure. Clinical practice guidelines emphasize the need for accurate detection and differentiation of atrial arrhythmias for effective management and optimal device usage. The systems and methods described herein provide a more comprehensive and accurate system and method for detecting and differentiating atrial arrhythmias to, among other things, optimize resources of the ambulatory medical device and ambulatory medical device usage for the patient.

Heart sounds are recurring mechanical signals associated with cardiac vibrations or accelerations from blood flow through the heart or other cardiac movements with each cardiac cycle or interval and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle (LV) during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

The S4 heart sound, which reflects atrial contraction during sinus rhythm, is frequently not present during atrial fibrillation. Accordingly, in certain examples, a determination of whether the S4 heart sound is present or absent can be used to improve determinations of atrial fibrillation. For example, S4 morphology can be been used to determine if the S4 heart sound is present in an S4 window within a specific cardiac cycle, such as disclosed in the commonly assigned Thakur et al. U.S. patent application Ser. No. 16/215,230, titled “Systems And Methods For Detecting Atrial Tachyarrhythmia Using Heart Sounds” (herein, “the '230 application”), hereby incorporated by reference in its entirety, including its disclosure of comparing a fourth heart sound (S4) signal portion to an S4 template and determining if a matching score exceeds a threshold value, and using the determined score to improve determinations of atrial fibrillation. In contrast, during non-fibrillation atrial arrhythmias, S4 amplitude can be amplified as atrial contraction remains somewhat organized.

Modified ventricular filling dynamics during atrial arrhythmias, such as during periods where ventricular rate is slower or more normalized causing the atria to contract against a more filled or stiff ventricle, can impact S4 amplitude. Inefficient ventricular filling can trigger a more forceful atrial contraction, resulting in an enhanced atrial contraction. In addition, underlying cardiac pathologies may cause both the rhythm disturbance and conditions of ventricular stiffness or hypertrophy.

Accordingly, changes in S4 amplitude and timing can provide valuable information for differentiating between various atrial arrhythmias. By analyzing the characteristics of the S4 heart sound in conjunction with other physiologic information, such as electrocardiogram data, the systems and methods described herein can improve the accuracy of atrial arrhythmia detection and classification. This approach addresses the limitations of conventional electrocardiogram-based approaches and may enhance the efficiency of cardiac monitoring devices by reducing false positives and optimizing resource utilization.

The cardiac cycle can be separated into a systolic interval, occurring when the ventricles contract to pump blood out of the heart, and a diastolic interval, occurring when the relaxes and the ventricles fill with blood from the atria. The systolic interval starts with the R wave or S1 heart sound at the closure of the mitral and tricuspid valves and ends at the S2 heart sound with the closure of the aortic and pulmonary valves. The diastolic interval begins with the S2 heart sound and ends at the subsequent R wave or S1 heart sound as the mitral and tricuspid valves close again. Diastole lasts longer than systole at normal heart rates, allowing adequate time for ventricular filling, but shortens significantly during faster heart rates.

$$Systolic Interval = R wave ( or S 1 location ) - S 2 location \qquad ( 1 )$$

$$Diastolic Interval = Cardiac cycle - Systolic Interval \qquad ( 2 )$$

In addition to changes in a S4 amplitude and timing, the diastolic interval shortens during atrial arrhythmias, which can severely compromise diastolic filling. This phenomenon manifests differently in various types of atrial arrhythmias, providing valuable information for their detection and differentiation.

During atrial fibrillation, the heart rate is highly irregular. This irregularity can lead to a reduction in the duration of some, but not all, diastolic intervals. The chaotic electrical activity characteristic of atrial fibrillation results in inconsistent ventricular filling times, which can be detected through analysis of diastolic intervals.

In contrast, during non-fibrillation atrial arrhythmias such as atrial flutter or atrial tachycardia, atrial contraction remains comparatively organized. As a result, the shortening of diastolic intervals in atrial flutter or atrial tachycardia are comparatively more consistent and pronounced in contrast to atrial fibrillation. This consistent shortening of diastolic intervals can serve as a distinguishing feature when analyzing cardiac cycles in these arrhythmias.

Differences in diastolic interval patterns can be used to enhance the detection and differentiation of atrial arrhythmias. By incorporating diastolic interval analysis with electrocardiogram and heart sound measurements, the systems and methods described herein can provide a more comprehensive approach to arrhythmia detection. This multi-parameter analysis can potentially improve the accuracy of diagnosis and monitoring of clinically significant atrial arrhythmias, addressing the limitations of conventional electrocardiogram-based approaches, and optimizing resources of the ambulatory medical device and ambulatory medical device usage for the patient.

Changes in several features derived from heart sounds can be utilized to differentiate various types of atrial arrhythmias and normal sinus rhythm. These differentiations include distinguishing atrial tachycardia from normal sinus rhythm, which can be used to confirm electrocardiogram-based atrial tachycardia episodes. This differentiation addresses the challenge of accurately identifying atrial tachycardia, which can be difficult to distinguish from normal sinus rhythm due to its regular heart rate pattern.

Additionally, the systems and methods described herein can differentiate symptomatic atrial arrhythmia (e.g., symptomatic, with symptoms, as opposed to asymptomatic, without symptoms) from normal sinus rhythm. This capability enhances the detection of clinically significant arrhythmias that may require intervention. The changes in heart sound features during atrial fibrillation, such as the diminished or absent S4, can be used to detect symptomatic episodes.

Furthermore, the systems and methods can detect symptomatic atrial tachycardia. The consistent and pronounced changes in heart sound features during atrial tachycardia can be leveraged to identify symptomatic episodes. The value of changes in heart sound features can be utilized to confirm or distinguish symptomatic atrial arrhythmias. This relationship provides a potential means to assess the clinical significance of detected arrhythmias, which can inform treatment decisions, patient management strategies, and control, programming, or utilization of ambulatory medical device and medical system resources.

For example, a detected increase in S4 (e.g., an increase in S4 amplitude) with a concurrent decrease in diastolic interval can be indicative of a high atrial tachycardia burden. In contrast, a detected decrease in S4 (e.g., a decrease in S4 amplitude) with a concurrent decrease in diastolic interval can be indicative of a high atrial fibrillation burden. The decrease in diastolic interval during atrial fibrillation episodes is often less than during corresponding atrial tachycardia episodes. These distinct patterns in S4 amplitude and diastolic interval changes provide valuable differentiating features between atrial tachycardia and atrial fibrillation episodes.

Additionally, distinct patterns in heart sound features can be used to differentiate different atrial arrhythmias, including specific changes or characteristics in S1 and S2 heart sounds. For example, S1 heart sound and S2 heart sound exhibit a decrease in amplitude at onset of atrial fibrillation. In contrast, at onset of atrial tachycardia, while S1 heart sounds exhibit a decrease in amplitude, S2 heart sounds can exhibit an increase in amplitude or remain substantially unchanged, providing a differentiating characteristic with respect to atrial fibrillation. These distinct patterns in S1 and S2 amplitude changes offer valuable differentiating features between atrial fibrillation and atrial tachycardia episodes.

The following changes can be used, separately or in different permutations or combinations, to distinguish between atrial tachycardia and atrial fibrillation, or between normal sinus rhythm and atrial tachycardia or atrial fibrillation. For atrial tachycardia, S1 heart sounds decrease, S2 heart sounds increase or remain unchanged, S4 heart sounds increase (e.g., substantially increase), and diastolic intervals decrease (e.g., substantially decrease). For atrial fibrillation, S1 heart sounds decrease (e.g., substantial decrease), S2 heart sounds decrease (e.g., substantial decrease), S4 heart sounds decrease, and diastolic intervals decrease. The above changes can be used in isolation or combination as a composite score. For example, changes in S4 can be used separately from the other characteristics to distinguish between atrial tachycardia and atrial fibrillation or between normal sinus rhythm and atrial tachycardia or normal sinus rhythm and atrial fibrillation. However, a composite score combining S4 with one or more other changes or combinations of the other changes can improve detection sensitivity and specificity. An example composite measure is indicated below, with a, b, c, and d as variables allowing different combinations or permutations of diastolic interval, S4 heart sounds, pre-S1 value, post-S2 value, and post-S3 value.

Composite=a(DI)+b(S1)+c(S4)+d(pre-S1)+e(post-S2)+f(post-S3) (3)

In certain examples, the composite can include separate atrial fibrillation and atrial tachycardia or atrial flutter composites where the S4 measure and the pre-S1 measure can be inverted (e.g., using the reciprocal of such measures) to account for the different changes expected by the different types of arrhythmia episodes described above.

By incorporating these heart sound-derived features into arrhythmia detection, the systems and methods described herein offer a more comprehensive approach to identifying and differentiating atrial arrhythmias. This multi-parameter analysis, combining cardiac electrical (e.g., electrocardiogram) and cardiac mechanical (e.g., heart sound) information in the manner described herein, addresses the limitations of conventional electrocardiogram-based detection and has the potential to improve the accuracy of arrhythmia diagnosis and monitoring.

In addition, as the diastolic interval shortens during atrial arrhythmias or periods of higher than normal atrial arrhythmia burden (e.g., such as in contrast to patient specific baselines or more generally across patients), heart sounds following S1, such as S3 and S4, and sometimes S2, tend to move into the last portion (e.g., 200 ms, etc.) of the cardiac cycle or diastolic interval before a succeeding R wave or S1 marking the start of the following systolic interval and cardiac cycle. Accordingly, specific time periods instead of specific heart sounds can include energy content that can be used to determine or distinguish between atrial tachycardia and atrial fibrillation, or between normal sinus rhythm and atrial tachycardia or normal sinus rhythm and atrial fibrillation. The specific time periods can include, in various combinations or permutations, a post-S1 time period (e.g., starting after S1), a post-S2 time period, a time period starting with S3, a post-S3 time period, or one or more time periods accomplished triggering back from a subsequent R wave or S1 location (e.g., 100 ms before the R-wave, 150 ms before the R wave, 200 ms before the R wave, 250 ms before the R wave or S1, etc.).

The S1 and S2 heart sounds are detectable by amplitude as specific features in a heart sound waveform or signal. The S3 and S4 heart sounds, in contrast, are generally identified as an integrated energy content in specific windows of time triggered by other cardiac or heart sound features (e.g., a time period from S2, the R wave, 51, etc.).

Although heart sound windows, such as for the S3 or S4 heart sound, are frequently in the order of 100 ms or 200 ms during normal sinus rhythm (NSR), during periods of atrial arrhythmias (e.g., having a high atrial arrhythmia burden or periods of detected atrial fibrillation or atrial tachycardia, etc.), such windows are often truncated or reduced due to the high atrial or ventricular rate. For example, during atrial tachycardia, atrial rates are frequently between 150 and 250 bpm while ventricular rates are often between 100 and 150 bpm due to conduction through the AV node. During atrial fibrillation, atrial rates are frequently even higher, often above 300 bpm or higher, with ventricular rates often between 100 and 180 bpm. Rate control drugs, such as beta blockers, calcium channel blockers, etc., can reduce the ventricular rate during atrial fibrillation below 100 bpm. At a ventricular rate of 150 bpm, the length of the cardiac cycle is only 400 ms. If diastole is approximately two-thirds of the cardiac cycle, such period at 150 bpm is only 267 ms and includes the separate S2, S3, and S4 heart sounds, each with space corresponding to different movement of blood and corresponding valve activity throughout the cardiac cycle. Cardiac cycle length at rates above 150 bpm are smaller yet. Accordingly, at high atrial or ventricular rates, instead of determining different heart sounds of the energy of heart sound waveforms or signals at different heart sound windows, the energy content of the heart sound waveform or signal can be integrated or otherwise filtered and accumulated over the diastolic interval or a substantial or set portion of the diastolic interval and used to distinguish between atrial tachycardia and atrial fibrillation or between normal sinus rhythm and atrial tachycardia or normal sinus rhythm and atrial fibrillation.

While discussed herein as being either a pre-R-wave or pre-S1 window, as heart sound information is measured in the window, there are advantages of the window being S1-based and not R-wave based such that only heart sound information is required (and not cardiac electrical information) for acquisition of the heart sound information. S1 and S2 are detectable in the heart sound information itself, separately from cardiac electrical information identification of an R wave, and the S3 and S4 windows can be based on S1-S1 or other cycle length duration (e.g., S2-S2, etc.) and identification of the S2 in the heart sound information.

In contrast, using the R wave or cardiac electrical information to trigger heart sound measurements can be advantageous in that the heart sound sensor (e.g., an accelerometer, etc.) can be triggered for activation only during the required window, such that the sensor is not required to be active for the entire cardiac cycle, only the required window. When looking back from a subsequent R wave, the previous cycle length or a range from a previous number of cycle lengths can be used to determine a likely next detection window to be trimmed or adjusted following detection of the R wave, saving power in contrast to continuous use or detection from the heart sound sensor.

In certain examples, a fixed duration (e.g., 100 ms, 150 ms, 200 ms, etc.) before the R wave or S1 can be reasonably expected to fully capture the S4 heart sound, regardless of atrial or ventricular rate. Additionally, triggering at a specific time reduces the need to set an S4 window based on any dynamic heart sound activity, such as a configurable period based on heart rate, detection and location of an S2 or determination of an S3 or other window, etc. The fixed duration can be heart rate agnostic, reducing required processing resources and providing a robust measurement standard for comparison. In addition, the fixed duration can capture other sounds (e.g., S2, S3, S4) that move into the window at high rates and can be impacted in different ways depending on different rhythms (e.g., normal sinus rhythm versus atrial fibrillation versus atrial flutter, etc.). Accordingly, a change in root means square (RMS) energy or other measurement of energy in the fixed duration window will change with different rhythms regardless of rate, providing valuable information for detection, determination, and in certain examples additional programming or operation.

In contrast, measuring all energy content in a dynamic window starting at the end of the S3 window through the R wave or S1 will specifically exclude all non-S4 heart sounds, providing a specific metric separate from S4 alone, ensuring S4 detection. In other examples, the dynamic window can be alternatively determined by heart rate or cycle length, from the diastolic interval (e.g., the final ⅔ of the diastolic window, etc.), from the start of an S3 window, each capturing all or part of the S3 and the full S4, ensuring capture of S3 and S4 merger at higher heart rates.

The present inventors have further recognized that the detection of heart sounds can be triggered by a determined arrhythmia burden (e.g., an atrial arrhythmia burden, atrial tachycardia burden, atrial fibrillation burden, atrial flutter burden, etc.) exceeding a threshold, by detection of one or more corresponding arrhythmia episodes, or by detection of one or more arrhythmia episodes at a specific burden (e.g., a low burden, a high burden, etc.). For example, an arrhythmia burden, such as a daily burden or a burden of one or more other time periods of one or more types of arrhythmia (e.g., an atrial tachycardia burden, an atrial fibrillation burden, or more generally an arrhythmia burden, etc.), can be determined for a patient using physiologic information, such as information indicative of arrhythmia episodes, etc., to provide a measure of the condition of the patient over the respective time period. At a high burden, such as relative to a baseline for the patient or one or more other clinical thresholds, heart sounds may be detected throughout the period that the determined burden is above the threshold. Triggering detection during a detected arrhythmia episode or otherwise during a corresponding high arrhythmia burden (e.g., a burden above a threshold) can reduce the likelihood of a false detection and unnecessary use of ambulatory device resources (e.g., for detection, storage, transmission, or subsequent processing, etc.). If heart sounds are detected throughout the period of high burden, during periods of low burden, upon detection of an arrhythmia episode, detection of heart sounds can be triggered for specific durations such as to verify, identify, or distinguish the detected arrhythmia. For example, triggering sensing of or receiving heart sound information, specifically to identify or confirm a high S4 heart sound measurement upon the detection of an atrial tachycardia episode, can provide confirmation of atrial tachycardia or atrial flutter.

Additionally, cardiac electrical information detection and collection, such as electrocardiogram recordings having a higher sampling rate than basic cardiac electrical information such as heart rate, etc., can be triggered by one or more heart sound measurements (e.g., S4 value, a daily average or high S4 value, a fixed or dynamic window value, etc.) exceeding one or more corresponding patient-specific or clinical thresholds (e.g., an absolute change or relative percentage change from a corresponding value at normal sinus rhythm, etc.). In other examples, heart sounds measurements can be triggered in response to a detected atrial fibrillation episode, and a low S4 or fixed or dynamic window value can be used to prioritize storage, transmission, or presentation of atrial fibrillation episodes for clinician review, or otherwise provide confirmation or adjudication of the detected episode for other programming or operation changes to the ambulatory medical device, etc.

Further, when therapy includes ablation, such as for atrial fibrillation or atrial flutter, etc., a change in detected heart sound information can be used to provide feedback on the impact of the therapy or used to guide or trigger additional follow-up (e.g., in-clinic follow-up) or therapy (e.g., additional ablation, etc.).

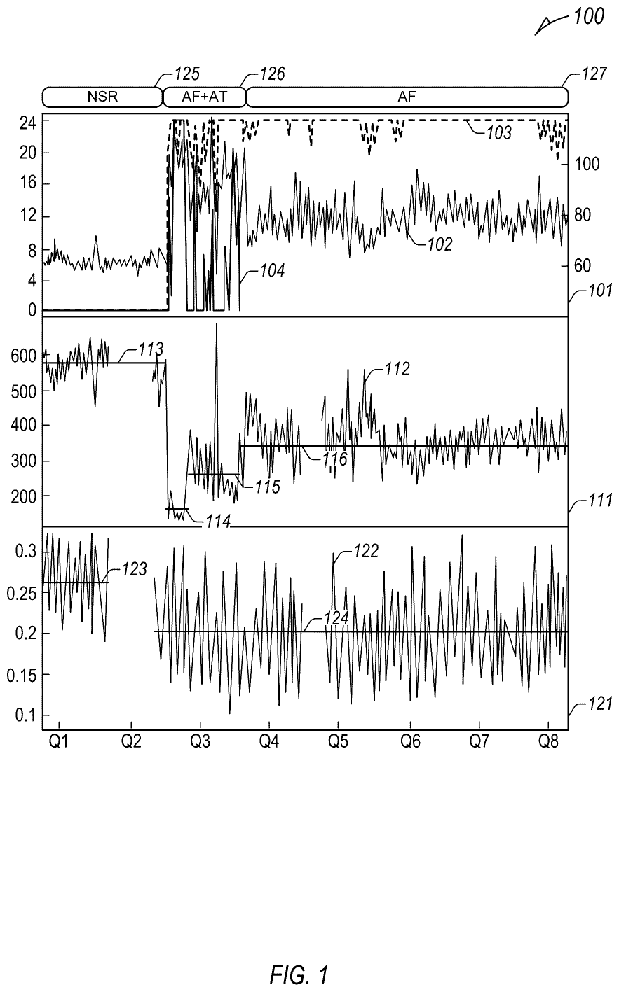

FIG. 1 illustrates a relationship 100 between physiologic parameters over time (in quarters Q1-Q8), including cardiac electrical information 101, diastolic interval (DI) information 111, and daily S4 information 121, highlighting the differentiation of the different physiologic parameters between different periods including a normal sinus rhythm (NSR) period 125, an atrial fibrillation and atrial tachycardia (AF+AT) period 126, and an atrial fibrillation (AF) period 127.

The cardiac electrical information 101 includes average heart rate values 102, such as a series of daily average values between 55 and 120 bpm, in addition to a determined atrial fibrillation burden 103 and a determined atrial tachycardia burden 104. The atrial fibrillation burden 103 and the atrial tachycardia burden 104 are illustrated in hours per day with a detected episode. In other examples, the atrial fibrillation burden 103 and the atrial tachycardia burden 104 can include hours per day with detected episodes above a threshold amount or one or more other measures indicative of arrhythmia burden to the patient, etc.

The average heart rate values 102 are lowest in FIG. 1 during the NSR period 125, highest during the AF+AT period 126, and elevated (in contrast to the NSR period 125) during the AF period 127. Heart rate information can be used, such as by one or more detection or assessment circuits, in initial arrhythmia detection or determination.

The diastolic interval information 111 includes daily diastolic interval values 112, such as a series of daily average values between 100 ms and 700 ms, with different levels during the different periods. The diastolic interval information 111 is at a first diastolic interval level 113 at about 580 ms during the NSR period 125. Between Q2 and Q3, the diastolic interval information 111 falls to a second diastolic interval level 114 at about 160 ms, coincident with a high atrial tachycardia burden 104 and high daily average heart rate values 102, during a first portion of the AF+AT period 126, before rising to a third diastolic interval level 115 at about 250 ms during a second portion of the AF+AT period 126 with a lower atrial tachycardia burden 104. Between Q3 and Q4, the diastolic interval information 111 rises to a fourth diastolic interval level 116 at about 350 ms during the AF period 127, above the third diastolic interval level 115 but below the first diastolic interval level 113.

The S4 information 121 includes daily S4 values 122, such as a series of daily average values between 0.1 and 0.35 in relative units (e.g., milli-g (mG), etc.) with different levels during different periods. The S4 information 121 is at a first S4 level 123 at about 0.26 during the NSR period 125, and at a second S4 level 124 at about 0.2 during the AF+AT period 126 and the AF period 127, without substantial change between the AF+AT period 126 and the AF period 127 illustrated by the diastolic interval information 111.

In other examples, the daily values in FIG. 1 can include daily average values, or one or more other measures of daily values, such as a median, minimum, maximum, percentage amount (e.g., upper quartile, lower quartile, etc.), etc. Although illustrated using information of a single patient in FIG. 1, such information can be indicative of general relationships between baseline information during periods of normal sinus rhythm, atrial fibrillation, atrial tachycardia, etc.

The present inventors have further recognized that heart sound information exhibits different frequency response over different types of arrhythmias. For example, frequency analysis of heart sound information, such as of an ensemble average of heart sound information over a time period (e.g., 30 seconds, 60 seconds, etc., stacked and aligned by a feature, such as S1, R wave, etc.), can be assembled and used to distinguish atrial tachycardia from atrial fibrillation. During atrial tachycardia, the heart sound information over time, such as illustrated in an ensemble average of heart sound information, demonstrates a regular, almost sinusoidal frequency pattern, characterized by the presence of slow frequency domination (e.g., small variation or changes in frequency across the ensemble average, etc.). This regularity contrasts sharply with the chaotic and irregular frequency patterns observed during atrial fibrillation. Accordingly, by performing frequency analysis (e.g., Fourier transform, short-time Fourier transform, wavelet transform, power spectral density analysis, etc.) on the heart sound information (e.g., the ensemble average heart sound information, such as over a 30-second window, etc.), one or more circuits or methods can identify the characteristic slow frequency resonance associated with atrial tachycardia or the chaotic irregular frequency patterns associated with atrial fibrillation, such as by determining a value of frequency density resulting from frequency analysis of the ensemble average heart sound information. The frequency analysis of the heart sound information, therefore, provides a robust mechanism for differentiating between atrial fibrillation and atrial tachycardia, enhancing the accuracy of arrhythmia detection and classification.

In an example, different frequency templates or thresholds can be determined or values of resonance or determined values of frequency density after frequency analysis for different cardiac rhythms, either specific to the patient or clinical values across patient populations, including normal sinus rhythm, atrial fibrillation, atrial tachycardia, atrial flutter, etc. In an example, the percent change from the normal sinus rhythm template or a baseline of different measures of resonance or frequency density after frequency analysis for the patient or a clinical population can be determined and compared to changes in the patient during a detected arrhythmia episode to distinguish between atrial tachycardia and atrial fibrillation, with atrial fibrillation more irregular and chaotic, having less density and more spread than atrial tachycardia. In an example, atrial fibrillation can deviate from a normal sinus rhythm or other baseline such as by a factor of 2× or greater than atrial tachycardia, the opposite of that illustrated in FIG. 1 with respect to diastolic interval, where atrial tachycardia illustrates a greater change than atrial fibrillation.

Further, the use of specific thresholds in the frequency analysis can enhance the differentiation between atrial tachycardia and atrial fibrillation. For example, during atrial tachycardia, the dominant frequency component should exceed a relative threshold value indicative of a significant presence of slow frequency components. In contrast, during atrial fibrillation, the dominant frequency component should exceed a relative threshold value indicative of a significant presence of higher frequency components. Additionally, a regularity index can be calculated to quantify the regularity of the frequency pattern. For atrial tachycardia, the regularity index should exceed a relative threshold value indicating a highly regular pattern. For atrial fibrillation, the regularity index should be below a relative threshold value indicating a highly irregular pattern.

In an example, information from the frequency analysis, such as a value of frequency density resulting from frequency analysis of the ensemble average heart sound or a change from a template or baseline, can be combined with any of the systems and methods described herein, in certain examples also part of a composite measure, in combination with one or more other features to determine the type of arrhythmia episode, or in the case of atrial fibrillation, to confirm the detected arrhythmia episode.

### Method Examples

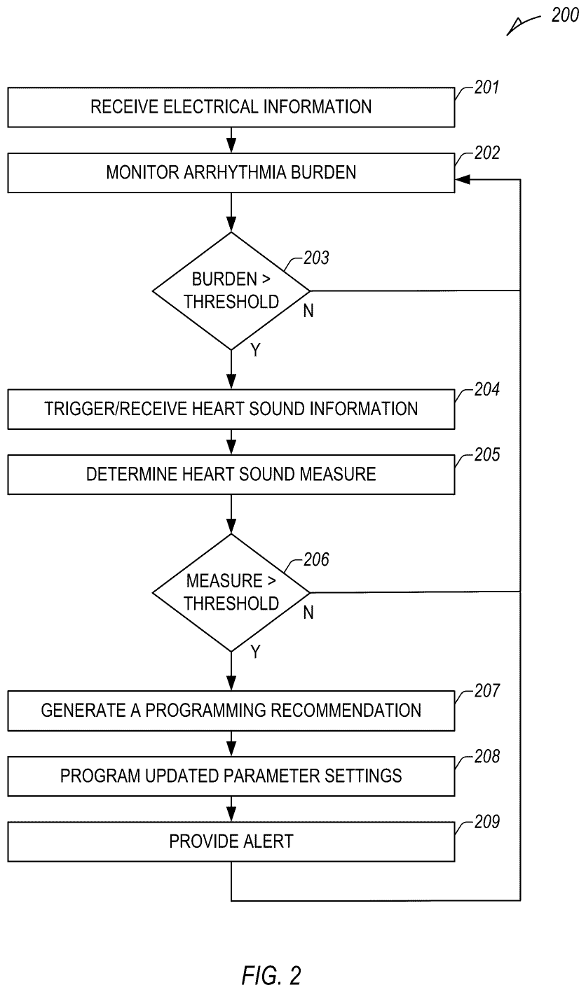

FIG. 2 illustrates a method 200 for improving arrhythmia detection by a medical device system to optimize resources of the medical device system. The medical device system can include an ambulatory medical device or one or more other components, such as illustrated here. The method 200 can be implemented using one or more processors and one or more memory devices storing instructions or using one or more circuits or components as described herein.

At step 201, electrical information of a patient can be received, such as using a signal receiver circuit or one or more other components. The electrical information can include cardiac electrical information of the patient, such as electrocardiogram or heart rate information of the patient, sensed or detected by one or more sensors, such as a cardiac electrical sensor, one or more electrodes and detection or sensor circuits, etc.

At step 202, arrhythmia burden of the patient can be determined and monitored, such as by an assessment circuit or one or more other components. In certain examples, cardiac electrical information of the patient can be used to detect an arrhythmia episode, such as atrial fibrillation, atrial tachycardia, atrial flutter, or one or more other types of atrial or ventricular arrhythmias. An arrhythmia burden, such as an atrial tachycardia burden, an atrial fibrillation burden, etc., can be determined as a number of detected episodes over time, or an amount of time associated with detected episodes relative to or over different time periods (e.g., hours per day with a detected episode, a number of detected episodes, or an amount of time associated with detected episodes over a threshold, etc.).

At step 203, the determined arrhythmia burden can be compared to a respective threshold, such as by the assessment circuit or one or more other components. In certain examples, the threshold can include a relative threshold based on a patient baseline (e.g., percent change from previous baseline or one or more previous time periods, etc.), a clinical threshold (e.g., received from or set by a clinician, etc.), a population threshold based on information from multiple patients, etc. If the determined arrhythmia burden exceeds the threshold, one or more actions can be triggered at step 204, such as sensing, receiving, or communicating heart sound information, etc.

For example, a duration of heart sound information (e.g., 30 seconds, etc.) can be sensed, such as by a heart sound sensor (e.g., an accelerometer), every 20 minutes, such as for determination of an ensemble average heart sound, to compare heart sound information, etc. In an example, the frequency of heart sound detection can be increased or decreased, such as with respect to the determined arrhythmia burden (e.g., a low burden can increase the time between detections, a high burden can reduce the time between detections, etc.). A change in the determined burden, such as with respect to a detected episode, exceeding a specific threshold can trigger sensing, reception, or communication of cardiac mechanical information at step 204, such as by the heart sound sensor or one or more communication circuits out of an ambulatory medical device to a remote programmer, etc.

At step 205, a heart sound measure can be determined, such as by the assessment circuit or one or more other components, using heart sound information. The heart sound measure can include one or more different parameters or combinations or permutations of different parameters, including, but not limited to, one or more of: S1 value, S2 value, S4 value, diastolic interval information, pre-S1 or pre-R-wave information, post-S2 information, post-S3 information, frequency analysis output (e.g., a determined value of frequency density, etc.), etc. In certain examples, the determined heart sound measure can include a determined composite score of multiple parameters.

In an example, the S1 value or the S2 value can be determined as an amplitude of a detected S1 heart sound or S2 heart sound, or as a value representative of an energy in a window centered about the S1 heart sound or the S2 heart sound. The S4 value, in contrast to the S1 value, can include a value of the heart sound information (e.g., an integrated energy) occurring over an S4 window of a first cardiac cycle (or first ensemble average) and ending at the beginning of a second cardiac cycle (or second ensemble average) immediately following the first cardiac cycle at a subsequent R wave or S1 heart sound.

Although discussed herein with respect a cardiac cycle, it is understood that the width of the cardiac cycle and the end, by reference to a beginning of a subsequent cardiac cycle, can likewise refer to the end of an ensemble average, which can itself include the beginning of a second cardiac cycle or alternatively reference a beginning of a second ensemble average following the first.

The diastolic interval can be determined using physiologic information (e.g., heart sound information) from a single cardiac cycle or across multiple cardiac cycles, such as an ensemble average of multiple cardiac cycles, etc., and can include a time period beginning at the beginning of the S2 heart sound of a first cardiac cycle and ending at the beginning of a second cardiac cycle immediately following the first cardiac cycle at a subsequent R wave or S1 heart sound.

The pre-S1 heart sound or pre-R-wave value can include a value of the heart sound information occurring over a respective pre-S1 heart sound or pre-R-wave period of time having a pre-determined duration occurring over a final portion of a first cardiac cycle and ending at the beginning of a second cardiac cycle immediately following the first cardiac cycle at a subsequent R wave or S1 heart sound. In an example, the pre-determined duration can be between 100 ms and 250 ms (e.g., 100 ms, 150 ms, 200 ms, 250 ms, etc.) or one or more other values to capture heart sound information at the end of the diastolic period, including merged hearts sounds occurring at higher heart rates, etc. For example, the pre-S1 heart sound may include a portion of one or several standard heart sounds, such as the S4 heart sound and the S3 heart sound, or one or more other combinations, etc. In other examples, the pre-determined duration can be less than 100 ms, for example, as little as 25 ms (e.g., between 25 ms and 100 ms, between 25 ms and 250 ms, etc.), such as to cover the S4 heart sound or include the S4 window, etc. Changes in the value associated with the pre-determined duration can be used to confirm a detected arrhythmia episode or to determine the type of the detected arrhythmia episode, for example, to distinguish between atrial tachycardia and atrial fibrillation, etc.

The post-S2 heart sound value can include a value of the heart sound information occurring over a post-S2 heart sound time period beginning at one of the end of the S2 heart sound or beginning or the end of the S3 heart sound or the S3 heart sound window of a first cardiac cycle and ending at the beginning of a second cardiac cycle immediately following the first cardiac cycle at a subsequent R wave or S1 heart sound. Changes in the post-S2 heart sound value, or similarly a post-S3 heart sound value, can be used to confirm a detected arrhythmia episode or to determine the type of the detected arrhythmia episode, for example, to distinguish between atrial tachycardia and atrial fibrillation, etc.

In other examples, the assessment circuit or one or more other components can be configured to perform frequency analysis on the heart sound information, such as an ensemble average of heart sound information over the detected arrhythmia episode determine a value of frequency density of the ensemble average of heart sound information. The heart sound measure can include the value of frequency density or one or more other measures of the frequency analysis on the heart sound information or the ensemble average of heart sound information over the detected arrhythmia episode.

At step 206, the determined heart sound measure can be compared to a respective threshold for the determined heart sound measure. In certain examples, the respective threshold can include a relative threshold based on a patient baseline of the determined heart sound measure (e.g., percent change from previous baseline or one or more previous time periods, etc.), a clinical threshold (e.g., received from or set by a clinician, etc.), a population threshold based on information from multiple patients, etc.

For example, a reduction in the S4 heart sound value for the detected arrhythmia episode with respect to a baseline value exceeding a threshold amount can be used to confirm the detected arrhythmia episode and to determine the type of the detected arrhythmia episode as an atrial fibrillation episode. Other relationships described herein can be used to confirm a detected arrhythmia episode (e.g., a reduction in a diastolic interval, a reduction in S1, a lack of increase in S2, etc.) or to determine the type of confirmed arrhythmia episode (e.g., a decrease in S2 indicating atrial fibrillation, a decrease in S4 indicating atrial fibrillation, an increase in S4 indicating atrial tachycardia, etc.).

If the determined measure exceeds the threshold, one or more determinations can be made and one or more actions can be triggered. For example, an alert can be communicated for atrial fibrillation, atrial flutter or atrial tachycardia (in certain examples combined due to their similar nature). In other examples, recorded physiological information, such as triggered recorded electrocardiogram or heart sound information for the determined atrial flutter or atrial tachycardia episode, or in other examples atrial fibrillation, can be flagged and elevated or prioritized for display, transfer, storage, or communication. In an example, communicating flagged episodes first, or only, can reduce the amount of power used in communication by an ambulatory medical device, representing substantial reduction in power and corresponding improvements in device lifespan and performance.

At step 207, one or more programming recommendations can be generated, such as by the assessment circuit or one or more other components, based on the determined heart sound measure, etc. At step 208, one or more updated parameter settings can be programmed, such as by the assessment circuit or one or more other components, based on the generated one or more programming recommendations, to optimize resources of a medical device system, etc.

For example, one or more modes or functions of the assessment circuit or an implantable or ambulatory medical device can be optionally adjusted based on determined measure. For example, if the determined measure represents a change from previous patient status, such as an improving patient status, one or more modes or functions of the implantable or ambulatory medical device can be altered to increase the remaining battery status of the medical device. In contrast, if the determined measure represents a worsening patient status, one or more modes or functions of the implantable or ambulatory medical device can be altered to improve data collection or sensing or to otherwise provide more patient benefit, reducing the remaining battery status of the medical device, but optimizing efficiency of the device with respect to the status of the patient. For example, one or more hardware limitations can be adjusted, such as to record more or less ECG information (e.g., requiring greater storage size, etc.) of the patient, increase communication frequency between the implantable or ambulatory medical device and an external device (e.g., remote device, programmer, etc.), increase the frequency of patient monitoring, switch to a different or more power or resource intensive monitoring algorithm, etc. In certain examples, one or more therapies can be optionally provided or adjusted based on the determined measure, such as described herein.

At step 209, an alert can be provided, such as by the assessment circuit or one or more other components, based on one or more of the steps above, etc. For example, an alert can be provided based on the burden exceeding the threshold at step 203, or the measure exceeding the threshold at step 206. In other examples, an alert can be provided if the determined measure or physiologic information from the detected episode having the determined measure exceeding the threshold are available for review or transmission to one or more other circuits or components. In an example, an output can be provided of the alert or the determined measure to a user interface for display to a user or to another circuit to control or adjust a process or a function of an implantable or ambulatory medical device, such as to adjust a therapy, mode, parameter settings, or follow-up schedule associated with the patient, a clinician, etc., with each of such adjustments at least partially dependent on one or more determined values, etc. In other examples, the determined measure or physiologic information from the detected episode having the determined measure exceeding the threshold can be stored, such as using the assessment circuit, and transmitted, by control of the assessment circuit or using one or more communication circuits, etc., such as to one or more additional processes or components, such as an output circuit (e.g., a display, a controller for a display, etc.).

Although illustrated as a series of steps above, in certain examples, one or more steps are optional, and in other examples, different combinations or permutations of these or other steps or examples can be combined to form other methods or processes, which is also applicable to other examples discussed herein.

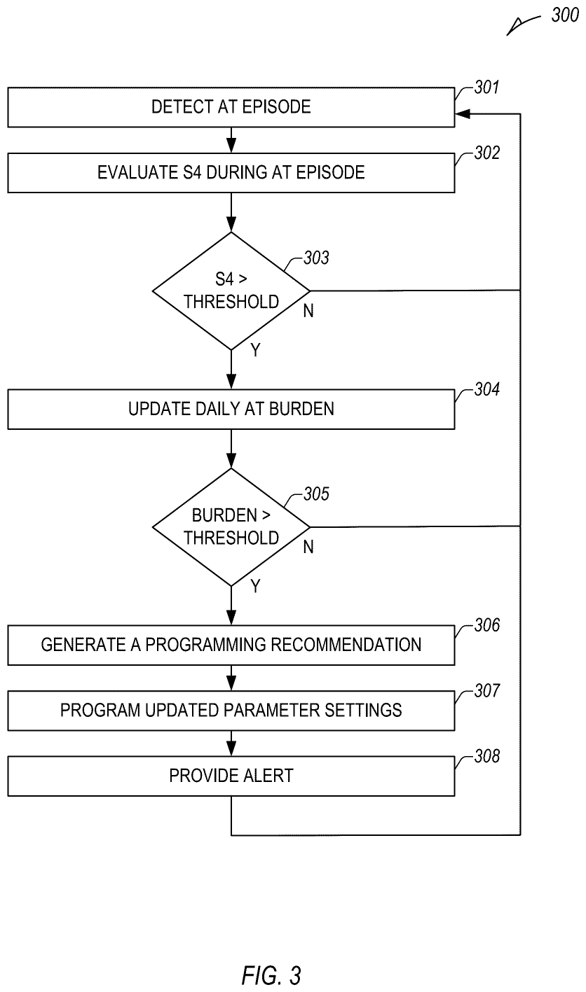

FIG. 3 illustrates a method 300 for improving arrhythmia detection, specifically atrial tachycardia, by a medical device system to optimize resources of the medical device system. The medical device system can include an ambulatory medical device or one or more other components, such as illustrated here. The method 300 can be implemented using one or more processors and one or more memory devices storing instructions or using one or more circuits or components as described herein.

At step 301, an atrial tachycardia episode (or atrial flutter or other atrial arrhythmia episode) can be detected, such as by an assessment circuit or one or more other components, using heart rate information of a patient, etc. In an example, detection of the atrial tachycardia episode can trigger sensing, receiving, communicating, or analyzing heart sound or other physiologic information, such as S4 information, diastolic interval, pre-S1 or pre-R-wave information, etc.

At step 302, heart sound information, such as S4 information during the detected atrial tachycardia episode, can be evaluated, such as by the assessment circuit or one or more other components. In an example, the S4 information can include an average S4 value during the detected atrial tachycardia episode or one or more other statistical S4 measures or other heart sound information, such as described herein.

At step 303, the value of the S4 information can be compared to a threshold, such as by the assessment circuit or one or more other components. The threshold can include a relative threshold based on a patient baseline (e.g., percent change from previous baseline or one or more previous time periods, etc.), a clinical threshold (e.g., received from or set by a clinician, etc.), a population threshold based on information from multiple patients, etc. If the value of the S4 information exceeds the threshold, one or more actions can be performed at step 304.

At step 304, an arrhythmia burden, such as a daily atrial tachycardia burden, can be determined or updated, such as by the assessment circuit or one or more other components. The arrhythmia burden can be determined or updated based on the evaluation of the S4 information during the detected atrial tachycardia episode at step 302 and the comparison to the threshold at step 303.

At step 305, the arrhythmia burden or a value of the arrhythmia burden can be compared to a threshold, such as by the assessment circuit or one or more other components. The threshold can include a relative threshold based on a patient baseline (e.g., percent change from previous baseline or one or more previous time periods, etc.), a clinical threshold (e.g., received from or set by a clinician, etc.), a population threshold based on information from multiple patients, etc. If a value of the arrhythmia burden exceeds the threshold, one or more actions can be performed at step 306.

At step 306, one or more programming recommendations can be generated, such as by the assessment circuit or one or more other components, based on the determined arrhythmia burden, etc. At step 307, one or more updated parameter settings can be programmed, such as by the assessment circuit or one or more other components, based on the generated one or more programming recommendations, to optimize resources of a medical device system, etc. For example, one or more modes or functions of the assessment circuit or an implantable or ambulatory medical device can be optionally adjusted, such as described herein.

At step 308, an alert can be provided, such as by the assessment circuit or one or more other components, based on one or more of the steps above or otherwise as described herein, etc. In other examples, an output can be provided or determined information can be stored or transmitted, by control of the assessment circuit or using one or more communication circuits, etc., such as to one or more additional processes or components, such as an output circuit (e.g., a display, a controller for a display, etc.), or otherwise as described herein.

Although illustrated as a series of steps above, in certain examples, one or more steps are optional, and in other examples, different combinations or permutations of these or other steps or examples can be combined to form other methods or processes, which is also applicable to other examples discussed herein.

### Medical Device System

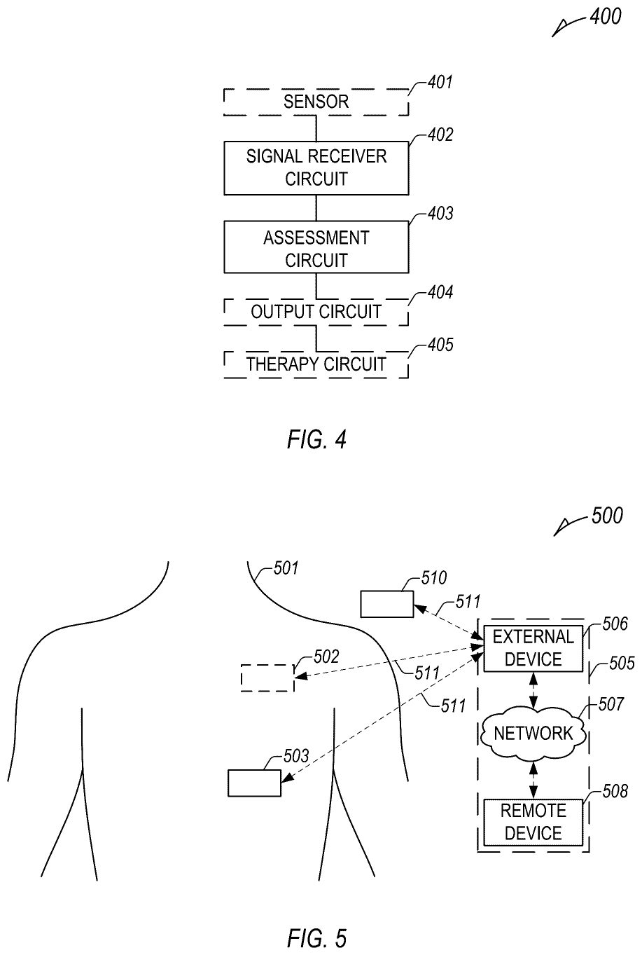

FIG. 4 illustrates an example system 400 (e.g., a medical device system). In an example, one or more aspects of the system 400 can be a component of, or communicatively coupled to, a medical device, such as an implantable medical device (IMD), an insertable cardiac monitor, an ambulatory medical device (AMD), etc. The system 400 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 400 can include a single medical device or a plurality of medical devices implanted in a body of a patient or otherwise positioned on or about the patient to monitor patient physiologic information of the patient using information from one or more sensors, such as a sensor 401. In an example, the sensor 401 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information, a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, a potassium sensor, a creatinine sensor, etc.); a temperature sensor; a skin elasticity sensor, or one or more other sensors configured to receive physiologic information of the patient.

The example system 400 can include a signal receiver circuit 402 and an assessment circuit 403. The signal receiver circuit 402 can be configured to receive physiologic information of a patient (or group of patients) from the sensor 401. The assessment circuit 403 can be configured to receive information from the signal receiver circuit 402, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. Physiologic information can include, among other things, one or more of: electrical information of the patient, such as cardiac electrical information (e.g., heart rate, heart rate variability, etc.), impedance information, temperature information, and in certain examples, respiration information (e.g., a respiratory rate, a respiration volume (tidal volume), etc.); mechanical information of the patient, such as cardiac acceleration information (e.g., cardiac vibration information, pressure waveform information, heart sound information, endocardial acceleration information, acceleration information, activity information, posture information, etc.), physical activity information (e.g., activity, steps, etc.), posture or position information, pressure information, plethysmograph information, and in certain examples, respiration information; chemical information; or other physiologic information of the patient. In an example, the signal receiver circuit 402 can include the sensor 401. In other examples, the signal receiver circuit can be coupled to or a component of the assessment circuit 403.

In certain examples, the assessment circuit 403 can aggregate information from multiple sensors or devices, detect various episodes using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event (such as a risk of a heart failure event, an arrhythmia episode, etc.), or to predict or stratify the risk of the patient experiencing an adverse medical event (e.g., a heart failure event, an arrhythmia episode, etc.) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 400 can include an output circuit 404 configured to provide an output to a user, or to cause an output to be provided to a user, such as through an output, a display, or one or more other user interface, the output including a score, a trend, an alert, or other indication. In other examples, the output circuit 404 can be configured to provide an output to another circuit, machine, or process, such as a therapy circuit 405 (e.g., a cardiac resynchronization therapy circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more cardiac resynchronization therapy parameters, drug delivery, dosage determinations or recommendations, etc. In an example, the therapy circuit 405 can include one or more of a stimulation control circuit, a cardiac stimulation circuit, a neural stimulation circuit, a dosage determination or control circuit, etc. In other examples, the therapy circuit 405 can be controlled by the assessment circuit 403, or one or more other circuits, etc. In certain examples, the assessment circuit 403 can include the output circuit 404 or can be configured to determine the output to be provided by the output circuit 404, while the output circuit 404 can provide the signals that cause the user interface to provide the output to the user based on the output determined by the assessment circuit 403.

### Efficiency and Mode Transitions

Ambulatory medical devices powered by rechargeable or non-rechargeable batteries, responsible for sensing physiologic signals and physiologic information of the patient, and in certain examples making determinations using such information, have to make certain tradeoffs between device battery life, or in the instance of implantable medical devices with non-rechargeable batteries, between device replacement periods often including surgical procedures, and device sensing, storage, processing, and communication characteristics, such as sensing resolution, sampling frequency, sampling periods, the number of active sensors, the amount of stored information, processing characteristics, or communication of physiologic information outside of the device.

Medical devices can include higher-power modes and lower-power modes. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode.

A technological problem in the art with respect to such devices exists that not all information can be stored, not all sensors can be active in a high-power or high-resolution mode, not all algorithms can be active, and not all sensed or processed information can be communicated outside of the device at all times without detrimentally impacting the lifespan of the devices. Technological solutions to such problems are often improvements in physical sensors, or alternatively in sensing and processing physiologic information in a way that improves device efficiency, extending the lifespan of the device, or to perform new determinations using existing sensors or information in a way that was not previously known, increasing the capabilities of an existing device without adding additional hardware to the device, or requiring additional sensors or hardware to be implanted in the patient. Efficiency improvements in one area can enable additional operation in another, improving the technical capabilities of existing devices having real-world constraints.

For example, physiologic information, such as indicative of a potential adverse physiologic event, can be used to transition from a low-power mode to a high-power mode. However, by the time physiologic information detected in the low-power mode indicates a possible event, valuable information has been lost, unable to be recorded in the high-power mode.

Another technological problem exists in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

In an example, a change in modes can enable higher resolution sampling or an increase in the sampling frequency or number or types of sensors used to sense physiologic information leading up to and including a potential event. Different physiologic information is often sensed using non-overlapping time periods of the same sensor, in certain examples, at different sampling frequencies and power costs.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected event (e.g., a heart failure event, an arrhythmia episode, etc.) can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

For example, ambulatory medical devices frequently contain one or more accelerometer sensors and corresponding processing circuits to determine and monitor patient acceleration information, such as, among other things, cardiac vibration information associated with blood flow or movement in the heart or patient vasculature (e.g., heart sounds, cardiac wall motion, etc.), patient physical activity or position information (e.g., patient posture, activity, etc.), respiration information (e.g., respiratory rate (RR), tidal volume (TV), rapid shallow breathing index (RSBI), respiration phase, breathing sounds, etc.), etc. In one example, heart sounds and patient activity can be detected using non-overlapping time periods of the same, single- or multi-axis accelerometer, at different sampling frequencies and power costs.

In an example, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

Another technological problem exists in that suboptimal programming of device parameters and parameter settings can negatively impact functionality of ambulatory medical devices. Accordingly, identifying suboptimal programming by clinicians and other caregivers and generating and providing alerts or notifications of such identified suboptimal programming, or reprogramming recommendations, and in certain examples, reprogramming ambulatory medical devices directly, can improve the functionality of existing ambulatory medical devices without requiring other improvements to the hardware of devices providing therapy or the sensors themselves.

### Implant and Follow-Up

When receiving a new medical device, patients may need to try several sets of parameter settings to receive sufficient or optimal therapy. In addition, in-clinic follow-up appointments are currently required as patient condition can change or response to existing devices or therapy can change over time, requiring additional changes to parameter settings that at one time were sufficient or optimal. In typical operation, a medical device, such as a cardiac resynchronization therapy device, is first programmed at a time of implant to a first operating mode, such as with respect to a first set of parameter settings, and then adjusted during scheduled in-clinic follow-up procedures with a clinician. Initial follow-up after implant (e.g., post-implant) or programming changes is generally a first time period, such as 4-6 weeks to allow patient recovery from the implant procedure and determination of baseline measurements for the patient from which to base future operation and monitoring, as well as to compare patient condition after implant or programming changes to the patient condition pre-implant or previous to programming changes. Subsequent follow-up after the initial follow-up can be less frequent, occurring, for example, every 3-6 months (e.g., at 6 months post-implant, at 12-months post-implant, etc.), or more or less as needed (e.g., between 1 and 12 months, etc.) depending on programming changes or changes in patient status or condition (e.g., a patient response metric). However, traditional follow-up appointments are in-person in a clinical setting and require travel for the patient, often at a substantial burden. In addition, programming changes may require additional follow-up, such as a new initial follow-up appointment and observation time, substantially increasing resources associated with programming the medical device and reducing the usable lifespan of the medical device during which the device is using limited resources to provide sufficient or optimal therapy.

In other examples, follow-up schedules can be determined or prioritized based on information from the ambulatory medical device, and moreover, changes can be made remotely, without in-person follow-up appointments, increasing the usable lifespan of the limited resources of the ambulatory medical device.

For implantable medical devices, once implanted and in certain examples after a recovery period, the implantable medical device can begin in a first mode, such as a cardiac resynchronization therapy mode, a monitoring mode, or one or more other therapy modes having different parameter settings, depending on the patient or clinician. In certain examples, a stimulation circuit can generate and provide one or more stimulation signals in one or more stimulation modes, and the assessment circuit can be configured to control the stimulation circuit, such as to adjust one or more parameters or transition between different therapy modes, etc.

### Physiologic Parameters

In certain examples, physiologic information of a patient can be sensed using one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. For example, the value and timing of an S1 signal can be detected using an amplitude or energy of the heart sound signal occurring at or about the R wave of the cardiac interval, and the value and timing of an S2 signal can be detected using an amplitude or energy of the heart sound signal occurring at or about the closure of the aortic and pulmonary valves, marking the transition from systole to diastole. S3 and S4 signal portions can be determined, such as by a processing circuit of the heart sound sensor or one or more other medical devices or medical device components, etc. In certain examples, the S3 signal portion can include a value or energy of the heart sound signal occurring in an S3 window of the cardiac interval, and the S4 signal portion can include a value or energy of the heart sound signal occurring in an S4 window of the cardiac interval. The S3 window occurs after S2 (e.g., 100 ms-200 ms after S2, 150 ms-200 ms after S2, etc.) and lasts for an S3 interval (e.g., 100 ms, 200 ms, etc.). The S4 window occurs shortly before the R wave or S1 (ending before or at the R wave or S1) and lasts for an S4 interval (e.g., 50 ms, 100 ms, 200 ms, etc.). The S3 or S4 windows and intervals can be determined as a set time period in the cardiac interval with respect to one or more other cardiac electrical or mechanical features, such as forward from one or more of the R wave, the T wave, or one or more features of a heart sound waveform or backwards from a subsequent R wave or a detected S1 of a subsequent cardiac interval. In certain examples, the length of the S3 or S4 intervals can depend on one or more factors, such as heart rate or patient characteristics, etc.

In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite first heart sound (S1) parameter representative of a plurality of S1 parameters, for example, over a certain time period (e.g., a number of cardiac cycles, a representative time period, etc.), or one or more other heart sounds (e.g., a second heart sound (S2), a third heart sound (S3), a fourth heart sound (S4), etc.), etc.

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform. In other examples, the signal receiver circuit can receive the at least one heart sound parameter or composite parameter, such as from a heart sound sensor or a heart sound sensor circuit.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient. In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In certain examples, additional physiologic information can be received, such as one or more of heart rate information, activity information of the patient, or posture information of the patient, from one or more other sensor or sensor circuits.

Respiration information can include, among other things, a respiratory rate (RR) of the patient, a tidal volume (TV) of the patient, a rapid shallow breathing index (RSBI) of the patient, or other respiratory information of the patient. The respiratory rate is a measure of a breathing rate of the patient, generally measured in breaths per minute. The tidal volume is an aggregate measure of respiration changes, such as detected using measured changes in thoracic impedance, etc. The RSBI is a measure (e.g., a ratio) of respiratory frequency relative to (e.g., divided by) tidal volume of the patient. The nHR is a measure of heart rate (HR) of the patient at night, either in relation to sensing patient sleep or using a preset or selectable time of day corresponding to patient sleep. In certain examples, respiration information of the patient can be determined using changes in impedance information and accordingly can be considered electrical information, but different than cardiac electrical information. In other examples, respiration information of the patient can be determined using changes in activity or acceleration information and accordingly can be considered mechanical information.

Physiologic metrics, as described herein, or measures or indications of physiologic information, can include one or more different measures of rate, amplitude, energy, etc., of different physiologic information over one or more time periods, such as representative daily values, etc. For example, heart sound metrics can be determined for each heart sound (e.g., the first heart sound (S1) through the fourth heart sound (S4), etc.) and can include an indication of an amplitude or energy of a specific heart sound for a specific cardiac cycle, or a representation of a number of cardiac cycles of the patient over a specific time period. Daily metrics can be determined representative of an average daily value for the patient, either corresponding to a waking time or a 24-hour period, etc. Respiration metrics can include, among other things, a mean or median respiratory rate, binned values of rates, and a representative value of specific rate bins, etc. Heart rate metrics can include an average nighttime heart rate, a minimum nighttime heart rate, heart rate at rest, etc.

The activity information can include an activity measurement of the patient, such as detected using an accelerometer, a posture sensor, a step counter, or one or more other activity sensors associated with an ambulatory medical device. Activity may be used to gate other physiologic measurements such as heart rate or respiratory rate so that the change in these metrics with increased patient activity may be used to infer patient cardiovascular and metabolic status including measurement of oxygen consumption. The impedance information can include, among other things, thoracic impedance information of the patient, such as a measure of impedance across a thorax of the patient from one or more electrodes associated with the ambulatory medical device (e.g., one or more leads of an implantable medical device proximate a heart of the patient and a housing of the implantable medical device implanted subcutaneously at a thoracic location of the patient, one or more external leads on a body of the patient, etc.). In other examples, the impedance information can include one or more other impedance measurements associated with the thorax of the patient, or otherwise indicative of patient thoracic impedance.

The temperature information can include an internal patient temperature at an ambulatory medical device, such as implanted in the thorax of the patient, or one or more other temperature measurements made at a specific location on the patient, etc. The temperature information can be detected using a temperature sensor, such as one or more circuits or electronic components having an electrical characteristic that changes with temperature. The temperature sensor can include a sensing element located on, at, or within the ambulatory medical device configured to determine a temperature indicative of patient temperature at the location of the ambulatory medical device.

In contrast to and separate from the electrical or mechanical information discussed above, the chemical information can include information about one or more chemical properties of blood, interstitial space (e.g., the space between cells, such as including interstitial fluid), or other tissue (e.g., muscle tissue, fat tissue, organ tissue, etc.) of the patient, such as information indicative of or including one or more of a glucose level, pH level, dissolved gas level (e.g. oxygen, carbon dioxide, carbon monoxide, etc.), electrolyte level (e.g., sodium, potassium, calcium, etc.), organic compound level (e.g., lactate, cholesterol, hemoglobin, creatinine, etc.), or biologic compound level (e.g., enzymes, antibodies, receptors, etc.), etc. The chemical information may be measured by one or more of an electrical sensor, mechanical sensor, electrochemical sensor, biosensor (e.g., enzyme biosensor, etc.), ion-selective electrode sensor, optical sensor, etc. In an example, the chemical information may include potassium information (e.g., one or more of interstitial potassium information, serum potassium information, etc.), creatinine information (e.g., one or more of interstitial creatinine information, serum creatinine information, etc.), or combinations thereof.

In certain examples, interstitial chemical information, such as one or more chemical levels in an interstitial space (e.g., a space between one or more of connective tissue, muscle fibers, nervous tissue, etc.) or of interstitial fluid, etc., can be indicative of serum chemical information. For example, potassium may move between cells or tissue and interstitial fluid (e.g., a change in interstitial potassium level may be followed by or reflective of a change in serum potassium level or vice versa), such that chemical information on serum potassium can include interstitial potassium. In certain examples, one of interstitial or serum chemical information can lead or lag the other, such that a change in one can indicate a worsening patient condition is detectable before the other. In one example, interstitial potassium information can lead serum potassium information as an indicator of electrolyte imbalance.

### Alert States

In certain examples, an alert state (e.g., an in-alert state, an out-of-alert state, a priority alert state, etc.) of the patient can be adjusted or determined using chemical information of the patient, such as to increase a sensitivity or specificity of alert state determination, reduce false positive alert state determinations, alert state transitions or adjustments, or otherwise reduce storage or transmission of physiologic information associated or transitions associated with false positive alert state determinations, and power and processing resources associated with the same. In an example, the alert state can be determined using a comparison of a value of the health index (e.g., a numerical value, etc.) to one or more fixed or adaptable alert thresholds (e.g., based at least in part on one or more relative factors, such as measurements from the patient over the past 30 days, etc.). In an example, the alert state can be provided to a user interface for display to a user or to a control circuit to control or adjust a process or function of the system. In an example, the alert state can include one or more of an indication, recommendation, or instruction to perform one or more actions (e.g., administer or provide a drug or class of drug, adjust or optimize a guideline-directed medical therapy (GDMT), etc.). For, example, a GDMT may advise administration of a quantity of a drug or a rate of increase in a dosage, etc. In an example, determination of an in-alert or priority alert state can trigger an indication or instruction to administer or provide a specific class of diuretic or to deviate from GDMT (e.g., increase GDMT above a standard recommendation, hold GDMT at a standard recommendation, hold GDMT at a current level, decrease GDMT below a standard recommendation, increase a dosage or rate of increase of a drug, reduce a dosage or rate of decrease of a drug, etc.).

In certain examples, the techniques described above or herein can be used in various combinations or permutations. For example, combinations or permutations of techniques described above or herein can be selected based upon patient history, patient treatment (e.g., in-patient care, out-patient care, etc.), clinician input, etc.

As used herein, high and low (or high, medium, and low, etc.) can be relative or categorical terms, in certain examples with respect to clinical or population values, patient-specific values (e.g., a representative value, such as a current value, with respect to a short- or long-term range of values, etc.), or combinations thereof. For example, a high value can include a value in an upper percentage (e.g., at or above an upper quartile, etc.) of values experienced by the patient over respective time periods, such as one or more of a short-term range (e.g., having a period between 1 week and 3 months, such as 1 month, etc.), a long term range (e.g., having a period greater than the short-term range, such as greater than 1 month, greater than 3 months, the last 6 months, or longer, etc.). A low value can include a value in a lower percentage (e.g., at or below a mean or median, below the upper quartile, etc.). A medium value can, in certain examples, include a value between the upper and lower quartiles or within a threshold percentage of a mean or median, etc. In other examples, values can be determined with respect to clinical or population values, in certain examples, further respective to matching patient demographics (e.g., age, sex, comorbidities, etc.) or type of medical device (e.g., CRT-D device, ICD device, etc.), etc.

In an example, determinations described herein can be used to change device behavior, trigger additional sensing, data processing, storage, or transmission, or otherwise alter one or more modes, processes, or functions of medical devices associated with such determinations. For example, determinations can require data over a substantial time period (e.g., multiple days, weeks, a month or more, etc.). Such determinations can be initially determined by the device at yearly or semi-yearly (e.g., every 6 months, every 3 months, etc.) by default, or triggered by worsening patient status or upon instruction from a clinician or caregiver, etc. In a first example, an assessment circuit can determine one or more indications quarterly, consuming a default amount of device resources. If the quarterly determination exceeds one or more of a patient-specific or population threshold, the assessment circuit can alter device functionality to increase the frequency of making such determinations, increasing the use of device resources, in certain examples reducing device lifespan, but providing additional monitoring and determinations. In other examples, if a determination exceeds one or more thresholds, additional sensing can be triggered, such as enabling additional sensors, or sensing enabled sensors with a higher resolution or sampling frequency, storing more information, and communicating more information outside of the device, such as to an external programmer, or increasing the frequency of communication outside of the device, increasing the use of device resources, in certain examples reducing device lifespan, but providing additional monitoring and determinations.

In certain examples, determinations described herein can include one or more determined risk curves illustrating determined risks at different time periods into the future, such as a determined risk of mortality (e.g., cardiovascular death), a determined risk of heart failure hospitalization, etc. Information about the determined risks or the determined risk curves or portions of the determined risk curves themselves can be provided to a user, such as to a patient, clinician, caregiver, etc., or can be used to make one or more device changes, such as described herein (e.g., therapies, treatments, device settings, etc.), or trigger one or more other processes or notifications, etc.

### Patient Indications

Indications of patient condition (e.g., improved or worsening patient condition, etc.) can include single-feature determinations based on a single feature or measure of a single type of physiologic information, or separately a composite determination based on a combination of physiologic information, such as two or more separate features of physiologic measures. In addition, indications of patient condition can be device-based, such as determined using physiologic information detected from the patient using the one or more ambulatory medical devices without input of clinical information about the patient separate from that detected or sensed physiologic information. In other examples, indications of patient condition can be a combination of device-based and clinical-based information of the patient, such as clinician diagnosis or determination of risk, patient history, patient age, comorbidities, prior hospitalization, type of implanted device, etc. In certain examples, separate determinations can be made for different combinations of clinical information.

One example of a composite indication is the HeartLogic™ index, a HeartLogic™ in-alert time, or one or more other composite measurements or measures thereof. The HeartLogic™ index is a composite indication of patient condition determined using different combinations or weightings of physiologic information, including two or more of S1 heart sounds, S3 heart sounds, thoracic impedance, activity information, respiration information, and nighttime heart rate (nHR). The HeartLogic™ index can be indicative of a heart failure status, a risk a heart failure event (e.g., within in a given time period), or a worsening of the heart failure status or risk of heart failure event in the patient over time. The HeartLogic™ in-alert time is a measure of time that the HeartLogic™ index is above an alert threshold.

In certain examples, the different combinations or weightings of physiologic information used to determine the HeartLogic™ index can be adjusted or determined based on a risk stratifier. In certain examples, the risk stratifier can be determined as a different combination of physiologic information, including one or more of S3, respiratory rate, and time active (e.g., an amount of time at a specific activity level above a mean activity level of the patient or a specific threshold, etc.). For example, if the risk stratifier is low, or below a first threshold, the HeartLogic™ index can be determined using a first combination of physiologic information. If the risk stratifier is high, or above a second threshold, the HeartLogic™ index can be determined using a second combination of physiologic information, such as additional information than included in the first combination (e.g., the first combination and the second combination, etc.). If the risk stratifier is between the first and second thresholds, the HeartLogic™ index can be determined using the first combination and one or more metrics or components of the second combination, or using the first combination and the second combination, but with the second combination having less weight than if the risk stratifier is above the second threshold (e.g., using less of the second combination than the first combination).

In an example, the HeartLogic™ index and in-alert time can include worsening heart failure or physiologic event detection, including risk indication or stratification, such as that disclosed in the commonly assigned An et al. U.S. Pat. No. 9,968,266 entitled “RISK STRATIFICATION BASED HEART FAILURE DETECTION ALGORITHM,” or in the commonly assigned An et al. U.S. Pat. No. 9,622,664 entitled “METHODS AND APPARATUS FOR DETECTING HEART FAILURE DECOMPENSATION EVENT AND STRATIFYING THE RISK OF THE SAME,” or in the commonly assigned Thakur et al. U.S. Pat. No. 10,660,577 entitled “SYSTEMS AND METHODS FOR DETECTING WORSENING HEART FAILURE,” or in the commonly assigned An et al. U.S. Patent Application No. 2014/0031643 entitled “HEART FAILURE PATIENT STRATIFICATION,” or in the commonly assigned Thakur et al. U.S. Pat. No. 10,085,696 entitled “DETECTION OF WORSENING HEART FAILURE EVENTS USING HEART SOUNDS,” each of which are hereby incorporated by reference in their entireties, including their disclosures of heart failure and worsening heart failure detection, heart failure risk indication detection, and stratification of the same, etc.

### Patient Management System

FIG. 5 illustrates an example patient management system 500 and portions of an environment in which the patient management system 500 may operate. The patient management system 500 can perform a range of activities, including remote patient monitoring and diagnosis of a disease condition, programming of ambulatory medical devices, and control of one or more therapies. Such activities can be performed proximal to a patient 501, such as in a patient home or office, through a centralized server, such as in a hospital, clinic, or physician office, or through a remote workstation, such as a secure wireless mobile computing device.

The patient management system 500 can include one or more medical devices, an external system 505, and a communication link 511 providing for communication between the one or more ambulatory medical devices and the external system 505. The one or more medical devices can include an ambulatory medical device, such as an implantable medical device 502, a wearable medical device 503, or one or more other implantable, leadless, subcutaneous, external, wearable, or medical devices configured to monitor, sense, or detect information from, determine physiologic information about, or provide one or more therapies to treat various conditions of the patient 501, such as one or more cardiac or non-cardiac conditions (e.g., dehydration, sleep disordered breathing, etc.).

In an example, the implantable medical device 502 can include one or more cardiac rhythm management (CRM) devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 501. In another example, the implantable medical device 502 can include a monitor implanted, for example, subcutaneously in the chest of patient 501, the implantable medical device 502 including a housing containing circuitry and, in certain examples, one or more sensors, such as a temperature sensor, etc.

Cardiac rhythm management devices are generally configured to receive cardiac electrical information from, and in certain examples, provide electrical stimulation to, one or more electrodes located within, on, or proximate to the heart, such as coupled to one or more leads and located in one or more chambers of the heart, within the vasculature of the heart near one or more chambers, or otherwise attached to or in contact with or proximate to the heart. Cardiac rhythm management devices can include, among others, pacemakers, implantable cardioverter defibrillators (ICD), subcutaneous implantable cardioverter defibrillators, cardiac resynchronization therapy defibrillators (CRT-Ds), insertable cardiac monitors, leadless cardiac pacemakers (LCPs), or wearable or remote monitoring systems.

Cardiac resynchronization therapy (CRT) refers generally to stimulation therapy generated and provided to one or more chambers of the heart (e.g., frequently two or more of the right ventricle (RV), the left ventricle (e.g., commonly through the cardiac vasculature), or the right atrium (RA), etc.) to improve cardiac function, such as to improve coordination of contractions between different chambers of the heart (e.g., the right ventricle and the left ventricle, the right atrium and the right ventricle, etc.) or to otherwise improve cardiac output or efficiency. Cardiac resynchronization therapy can include biventricular pacing (e.g., both right and left ventricular pacing), single-chamber pacing (e.g., right ventricle pacing, left ventricle pacing, etc.), sensing or pacing in one or more other chambers or combinations of chambers (e.g., right atria, etc.), as well as multi-site pacing (MSP) (e.g., applying one or more stimulation signals to multiple (e.g., two or more) electrodes in or proximate to a chamber (e.g., commonly the left ventricle, but also in certain examples the right ventricle, the right atrium, or combinations thereof) for a single cardiac cycle), and in certain examples, HIS-bundle pacing, septal pacing, etc. The timing of stimulation signals in the cardiac cycle or with respect to one or more cardiac events often varies depending on a number of factors, including placement of the lead or electrodes, propagation of the stimulation signals through the tissue, and stimulation parameters, such as stimulation amplitude, type, timing, etc.

Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured to detect physiologic information from the patient, or provide one or more therapies or stimulation to the patient.

Implantable devices can additionally or separately include leadless cardiac pacemakers, small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemaker devices can be implanted in or about the heart to detect physiologic information from, or provide one or more therapies or stimulation to, one or more chambers of the heart. The multiple leadless cardiac pacemaker devices can communicate between themselves, or one or more other implanted or external devices.

The implantable medical device 502 can include a signal receiver circuit or an assessment circuit configured to detect or determine specific physiologic information of the patient 501, or to determine one or more conditions or provide information or an alert to a user, such as the patient 501 (e.g., a patient), a clinician, or one or more other caregivers or processes, such as described herein. The implantable medical device 502 can alternatively or additionally be configured as a therapeutic device configured to treat one or more medical conditions of the patient 501. The therapy can be delivered to the patient 501 via the lead system and associated electrodes or using one or more other delivery mechanisms. The therapy can include delivery of one or more drugs to the patient 501, such as using the implantable medical device 502 or one or more of the other ambulatory medical devices, etc. In some examples, therapy can include cardiac resynchronization therapy for rectifying dyssynchrony and improving cardiac function in heart failure patients. In other examples, the implantable medical device 502 can include a drug delivery system, such as a drug infusion pump to deliver drugs to the patient for managing arrhythmias or complications from arrhythmias, hypertension, hypotension, or one or more other physiologic conditions. In other examples, the implantable medical device 502 can include one or more electrodes configured to stimulate the nervous system of the patient or to provide stimulation to the muscles of the patient airway, etc.

The wearable medical device 503 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 505 can include a dedicated hardware/software system, such as a programmer, a remote server-based patient management system, or alternatively a system defined predominantly by software running on a standard personal computer. The external system 505 can manage the patient 501 through the implantable medical device 502 or one or more other ambulatory medical devices connected to the external system 505 via a communication link 511. In other examples, the implantable medical device 502 can be connected to the wearable medical device 503, or the wearable medical device 503 can be connected to the external system 505, via the communication link 511. This can include, for example, programming or reprogramming the implantable medical device 502 with different parameter settings to perform one or more of acquiring physiologic data, performing at least one self-diagnostic test (such as for a device operational status), analyzing the physiologic data, or optionally delivering or adjusting a therapy for the patient 501. Additionally, the external system 505 can send information to, or receive information from, the implantable medical device 502 or the wearable medical device 503 via the communication link 511. Examples of the information can include real-time or stored physiologic data from the patient 501, diagnostic data, such as detection of patient hydration status, hospitalizations, responses to therapies delivered to the patient 501, or device operational status of the implantable medical device 502 or the wearable medical device 503 (e.g., battery status, lead impedance, etc.). The communication link 511 can be an inductive telemetry link, a capacitive telemetry link, or a radio frequency (RF) telemetry link, such as a wireless telemetry based on, for example, Bluetooth® or IEEE 802.11 wireless fidelity “Wi-Fi” interfacing standards. Other configurations and combinations of patient data source interfacing are possible.

The external system 505 can include an external device 506 in proximity of the one or more ambulatory medical devices, and a remote device 508 in a location relatively distant from the one or more ambulatory medical devices, in communication with the external device 506 via a communication network 507. Examples of the external device 506 can include a medical device programmer. The remote device 508 can be configured to evaluate collected device or patient information and provide alert notifications, among other possible functions. In an example, the remote device 508 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 508 can receive data from multiple patients. The data can be collected by the one or more ambulatory medical devices, among other data acquisition sensors or devices associated with the patient 501. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text, or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

In an example, similar to the alert notifications discussed above, the external system 505 or one or more components thereof (e.g., the external device 506, the remote device 508, an assessment circuit, etc.) can be configured to schedule one or more follow-up appointments or adjust a schedule of one or more follow-up appointments for the patient such as in response to one or more alert notifications or other determinations, per a request of a clinician, etc.

The remote device 508 may additionally include one or more locally configured clients or remote clients securely connected over the communication network 507 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 508, including the server and the interconnected clients, may also execute a follow-up scheme by sending follow-up requests to the one or more ambulatory medical devices, or by sending a message or other communication to the patient 501 (e.g., the patient), clinician or authorized third party as a compliance notification.

The communication network 507 can provide wired or wireless interconnectivity. In an example, the communication network 507 can be based on the Transmission Control Protocol/Internet Protocol (TCP/IP) network communication specification, although other types or combinations of networking implementations are possible. Similarly, other network topologies and arrangements are possible.

One or more of the external device 506 or the remote device 508 can output the detected medical events to a system user, such as the patient or a clinician, or to a process including, for example, an instance of a computer program executable in a microprocessor. In an example, the process can include an automated generation of a programming recommendation for an ambulatory medical device to optimize or improve patient condition or otherwise provide a desired clinical outcome. In an example, the external device 506 or the remote device 508 can include a respective display unit for displaying the physiologic or functional signals, or alerts, alarms, emergency calls, or other forms of warnings to signal the detection of one or more conditions. In some examples, the external system 505 can include a signal receiver circuit and an assessment circuit, such as an external data processor configured to analyze the physiologic or functional signals received by the one or more ambulatory medical devices, and to confirm or reject one or more determinations made by one or more ambulatory medical devices, such as the implantable medical device 502, the wearable medical device 503, etc., or make additional determinations, etc. Computationally intensive algorithms, such as machine-learning algorithms, can be implemented in the external data processor.

With some examples, when parameter settings of an ambulatory medical device are analyzed using one or more trained machine learning models, and one or more differences between the parameter settings of the ambulatory medical device and the stored model parameter settings are detected, a recommendation to reprogram the medical device may be generated and presented to a clinician via a user interface of the remote device 508, or via a user interface of a software application executing on a client device communicatively connected with the remote device 508. The recommendation to reprogram the medical device may be determined by identifying differences between the parameter settings of the ambulatory medical device and the stored model parameter settings via the one or more machine learning models that otherwise went undetected by a clinician or a medical device programmer.

Portions of the one or more ambulatory medical devices or the external system 505 can be implemented using hardware, software, firmware, or combinations thereof. Portions of the one or more ambulatory medical devices or the external system 505 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

A therapy device 510 can be configured to send information to or receive information from one or more of the ambulatory medical devices or the external system 505 using the communication link 511. In an example, the one or more ambulatory medical devices, the external device 506, or the remote device 508 can be configured to control one or more parameters of the therapy device 510. The external system 505 can allow for programming or reprogramming the one or more ambulatory medical devices and can receive information about one or more signals acquired by the one or more ambulatory medical devices, such as can be received via a communication link 511. The external system 505 can include a local external implantable medical device programmer. The external system 505 can include a remote patient management system that can monitor patient status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected events can be stored, such as to increase the specificity of detection. In an example, multiple loop recorder windows (e.g., 2-minute windows, 4-minute windows, etc.) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), in certain examples, in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, for example, to increase arterial pressure, to maintain cardiac output, to disrupt or reduce the impact of the detected event, or combinations thereof.

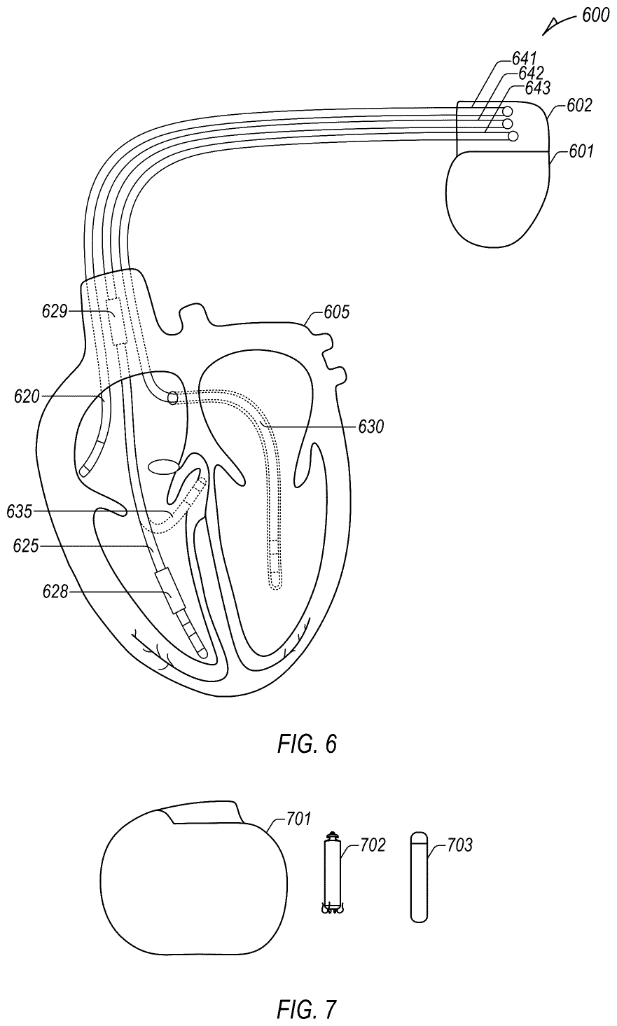

FIG. 6 illustrates an example implantable medical device system 600 including an implantable medical device 601 electrically coupled to a heart 605, such as through one or more leads coupled to the implantable medical device 601 through one or more lead ports, including first, second, or third lead ports 641, 642, 643 in a header 602 of the implantable medical device 601. In an example, the implantable medical device 601 can include an antenna, such as in the header 602, configured to enable communication with an external system and one or more electronic circuits (e.g., an assessment circuit, etc.) in a hermetically sealed housing (CAN).

The implantable medical device 601 may include an insertable cardiac monitor, pacemaker, defibrillator, cardiac resynchronization therapy device, or other subcutaneous implantable medical device or cardiac rhythm management device configured to be implanted in a chest of a patient, having one or more leads to position one or more electrodes or other sensors at various locations in or near the heart 605, such as in one or more of the atria or ventricles. Separate from, or in addition to, the one or more electrodes or other sensors of the leads, the implantable medical device system 600 can include one or more electrodes or other sensors (e.g., a pressure sensor, an accelerometer, a gyroscope, a microphone, etc.) powered by a power source in the implantable medical device 601. The one or more electrodes or other sensors of the leads, the implantable medical device 601, or a combination thereof, can be configured to detect physiologic information from, or provide one or more therapies or stimulation to, the patient.

Implantable devices can additionally include a leadless cardiac pacemaker, small (e.g., smaller than traditional implantable devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart 605 without traditional lead or implantable device complications (e.g., required incision and pocket, complications associated with lead placement, breakage, or migration, etc.). In certain examples, a leadless cardiac pacemaker can have more limited power and processing capabilities than a traditional CRM device; however, multiple leadless cardiac pacemaker devices can be implanted in or about the heart to detect physiologic information from, or provide one or more therapies or stimulation to, one or more chambers of the heart. The multiple leadless cardiac pacemaker devices can communicate between themselves, or one or more other implanted or external devices.

The implantable medical device 601 can include one or more electronic circuits configured to sense one or more physiologic signals, such as an electrogram or a signal representing mechanical function of the heart 605. In certain examples, the housing may function as an electrode such as for sensing or pulse delivery. For example, an electrode from one or more of the leads may be used together with the housing such as for unipolar sensing of an electrogram or for delivering one or more pacing pulses. A defibrillation electrode (e.g., the first defibrillation coil electrode 628, the second defibrillation coil electrode 629, etc.) may be used together with the housing to deliver one or more cardioversion/defibrillation pulses.

In an example, the implantable medical device 601 can sense impedance such as between electrodes located on one or more of the leads or the housing. The implantable medical device 601 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the implantable medical device 601 can be configured to inject current between an electrode on one or more of the first, second, third, or fourth leads 620, 625, 630, 635 and the housing, and to sense the resultant voltage between the same or different electrodes and the housing.

The implantable medical device 601 can integrate one or more other physiologic sensors to sense one or more other physiologic signals, such as one or more of heart rate, heart rate variability, thoracic or intrathoracic impedance, intracardiac impedance, arterial pressure, pulmonary artery pressure, right ventricular pressure, left ventricular coronary pressure, coronary blood temperature, blood oxygen saturation, one or more heart sounds, physical activity or exertion level, physiologic response to activity, posture, respiration, body weight, or body temperature. The arrangement and functions of these leads and electrodes are described above by way of example and not by way of limitation. Depending on the need of the patient and the capability of the implantable device, other arrangements and uses of these leads and electrodes are contemplated.

FIG. 7 illustrates example implantable medical devices, including a subcutaneous implantable cardioverter defibrillator 701, a leadless cardiac pacemaker 702 including tines for placement, and an insertable cardiac monitor 703.

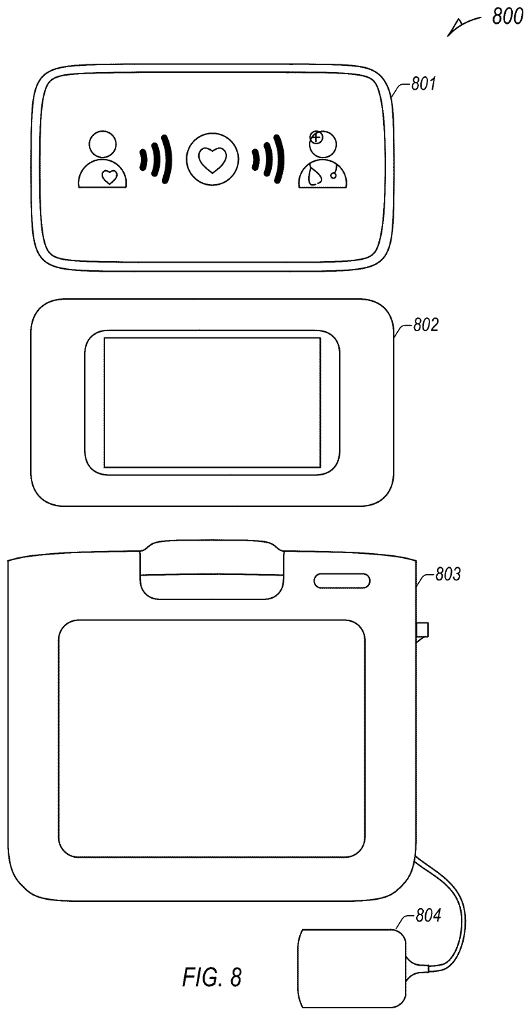

FIG. 8 illustrates example remote patient management systems 800 for communication with an ambulatory medical device, such as for receiving information from or providing information to, including programming instructions, one or more ambulatory medical devices. The example remote patient management systems 800 include a first remote patient management device 801 (e.g., a LATITUDE™ NXT Remote Patient Management System) for at-home monitoring and RF telemetry capabilities through one or more communication circuits with an ambulatory medical device and communication to a cloud-based server or clinician programming environment through a network connection, a second remote patient management device 802 (e.g., an EMBLEM™ S-ICD Programmer) with RF telemetry capabilities through one or more communication circuits and an optional external telemetry wand for communication with an ambulatory medical device, and a third remote patient management system 803 (e.g., a LATITUDE™ Programming System, Model 3300, etc.) with RF telemetry capabilities through one or more communication circuits and an external telemetry wand 804 (e.g., a Model 6395 Telemetry Wand, etc.) including an external telemetry coil configured for inductive communication with a corresponding telemetry coil of an implantable medical device. Although not illustrated herein, the remote patient management systems 800 can include one or more other remote patient management systems, such as one or more other LATITUDE™ Programming systems, a remote patient monitoring application for a mobile device of a patient or other caregiver, etc. Each type of remote patient monitoring or management system has different capabilities and in certain examples permissions with respect to different programming instructions or features.

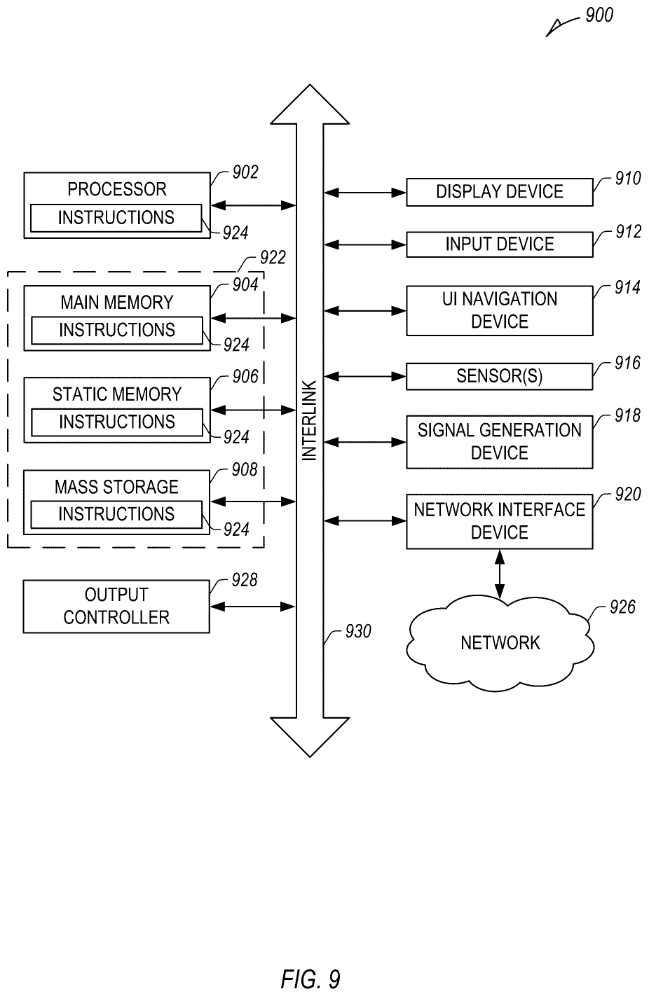

FIG. 9 illustrates a block diagram of an example machine 900 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 900. Circuitry (e.g., processing circuitry, an assessment circuit, etc.) is a collection of circuits implemented in tangible entities of the machine 900 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to perform a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to perform portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 900 follow.

In alternative embodiments, the machine 900 may operate as a standalone device or may be connected (e.g., networked) to other machines. In a networked deployment, the machine 900 may operate in the capacity of a server machine, a client machine, or both in server-client network environments. In an example, the machine 900 may function as a peer machine in peer-to-peer (P2P) (or other distributed) network environment. The machine 900 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 900 (e.g., computer system) may include a hardware processor 902 (e.g., a central processing unit (CPU), a graphics processing unit (GPU), a hardware processor core, or any combination thereof), a main memory 904, a static memory 906 (e.g., memory or storage for firmware, microcode, a basic-input-output (BIOS), unified extensible firmware interface (UEFI), etc.), and mass storage 908 (e.g., hard drive, tape drive, flash storage, or other block devices) some or all of which may communicate with each other via an interlink 930 (e.g., bus). The machine 900 may further include a display unit 910, an alphanumeric input device 912 (e.g., a keyboard), and a user interface (UI) navigation device 914 (e.g., a mouse). In an example, the display unit 910, input device 912, and UI navigation device 914 may be a touch screen display. The machine 900 may additionally include a signal generation device 918 (e.g., a speaker), a network interface device 920, and one or more sensors 916, such as a global positioning system (GPS) sensor, compass, accelerometer, or one or more other sensors. The machine 900 may include an output controller 928, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 902, the main memory 904, the static memory 906, or the mass storage 908 may be, or include, a machine-readable medium 922 on which is stored one or more sets of data structures or instructions 924 (e.g., software) embodying or utilized by any one or more of the techniques or functions described herein. The instructions 924 may also reside, completely or at least partially, within any of registers of the processor 902, the main memory 904, the static memory 906, or the mass storage 908 during execution thereof by the machine 900. In an example, one or any combination of the hardware processor 902, the main memory 904, the static memory 906, or the mass storage 908 may constitute the machine-readable medium 922. While the machine-readable medium 922 is illustrated as a single medium, the term “machine-readable medium” may include a single medium or multiple media (e.g., a centralized or distributed database, and/or associated caches and servers) configured to store the one or more instructions 924.

The term “machine-readable medium” may include any medium that is capable of storing, encoding, or carrying instructions for execution by the machine 900 and that cause the machine 900 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 924 may be further transmitted or received over a communications network 926 using a transmission medium via the network interface device 920 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 920 may include one or more physical jacks (e.g., Ethernet, coaxial, or phone jacks) or one or more antennas to connect to the communications network 926. In an example, the network interface device 920 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 900, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical device system for improving arrhythmia detection to optimize resources of the medical device system, comprising:
a signal receiver circuit configured to receive physiologic information of a patient, the physiologic information including cardiac electrical and cardiac mechanical information; and
an assessment circuit configured to:
detect an arrhythmia episode using the cardiac electrical information;
analyze the cardiac mechanical information occurring over a final portion of a cardiac cycle of the detected arrhythmia episode to confirm the detected arrhythmia episode or to determine a type of the detected arrhythmia episode based on analysis of the cardiac mechanical information; and
provide a control signal to control a mode or operation of the medical device system based on the confirmation of the detected arrhythmia episode or the determined type of the detected arrhythmia episode to optimize resources of the medical device system.

2. The medical device system of claim 1, wherein the assessment circuit is configured to trigger sensing of or receiving the cardiac mechanical information, including heart sound information, based on the detected arrhythmia episode.

3. The medical device system of claim 1, wherein the cardiac electrical information includes heart rate information,
wherein to analyze the cardiac mechanical information includes to confirm the detected arrhythmia episode and to determine the type of the detected arrhythmia episode based on the analysis of the cardiac mechanical information,
wherein to determine the type of arrhythmia episode includes to determine one of a plurality of types of arrhythmia episode including an atrial tachycardia episode, an atrial fibrillation episode, or an atrial flutter episode.

4. The medical device system of claim 3, wherein the assessment circuit is configured to trigger sampling and storage of an electrocardiogram signal at a sampling rate and storage size greater than the heart rate information in response to the cardiac mechanical information exceeding a threshold.

5. The medical device system of claim 1, wherein the cardiac mechanical information includes an S4 heart sound value,
wherein the assessment circuit is configured to determine and analyze the S4 heart sound value occurring over an S4 window of a first cardiac cycle and ending at a beginning of a second cardiac cycle immediately following the first cardiac cycle at a subsequent R wave or S1 heart sound.

6. The medical device system of claim 5, wherein the assessment circuit is configured to detect a change in the S4 heart sound value for the detected arrhythmia episode with respect to a baseline value and to confirm the detected arrhythmia episode and to determine the type of the detected arrhythmia episode based on the detected change.

7. The medical device system of claim 1, wherein the cardiac mechanical information includes a pre-S1 heart sound or pre-R-wave value,
wherein the assessment circuit is configured to determine and analyze the pre-S1 heart sound or the pre-R-wave value occurring over a respective pre-S1 heart sound or pre-R-wave period of time having a pre-determined duration occurring over a final portion of a first cardiac cycle and ending at a beginning of a second cardiac cycle immediately following the first cardiac cycle at a subsequent R wave or S1 heart sound,
wherein the pre-determined duration is between 100 ms and 250 ms,
wherein the assessment circuit is configured to confirm the detected arrhythmia episode and to determine the type of the detected arrhythmia episode based on the pre-S1 heart sound or the pre-R-wave value.

8. The medical device system of claim 1, wherein the cardiac mechanical information includes a post-S2 heart sound value,
wherein the assessment circuit is configured to determine and analyze the post-S2 heart sound value occurring over a post-S2 heart sound time period beginning at one of an end of an S2 heart sound or a beginning or an end of an S3 heart sound or an S3 heart sound window of a first cardiac cycle and ending at a beginning of a second cardiac cycle immediately following the first cardiac cycle at a subsequent R wave or S1 heart sound,
wherein the assessment circuit is configured to confirm the detected arrhythmia episode and to determine the type of the detected arrhythmia episode based on the post-S2 heart sound value.

9. The medical device system of claim 1, wherein the cardiac mechanical information includes a diastolic interval,
wherein the assessment circuit is configured to determine and analyze the diastolic interval as a time period beginning at a beginning of an S2 heart sound of a first cardiac cycle and ending at a beginning of a second cardiac cycle immediately following the first cardiac cycle at a subsequent R wave or S1 heart sound,
wherein the assessment circuit is configured to confirm the detected arrhythmia episode and to determine the type of the detected arrhythmia episode based on the diastolic interval.

10. The medical device system of claim 9, wherein the assessment circuit is configured to detect a reduction in the diastolic interval for the detected arrhythmia episode with respect to a baseline value and to determine the type of the detected arrhythmia episode based on the detected reduction exceeding a threshold.

11. The medical device system of claim 9, wherein the assessment circuit is configured to:
determine at least one of an S4 heart sound value, a pre-S1 heart sound or pre-R-wave value occurring over a pre-S1 heart sound or pre-R-wave period of time having a pre-determined duration of 100 ms occurring over a final portion of the first cardiac cycle and ending at the beginning of the second cardiac cycle, or a post-S2 heart sound value occurring over a post-S2 heart sound time period beginning at one of an end of the S2 heart sound or a beginning or an end of an S3 heart sound or an S3 heart sound window of the first cardiac cycle and ending at the beginning of the second cardiac cycle;
determine a composite cardiac mechanical measure as a function of the determined diastolic interval and the determined at least one of the S4 heart sound value, the pre-S1 heart sound or pre-R-wave value, or the post-S2 heart sound value; and
determine the type of the detected arrhythmia episode based on the determined composite cardiac mechanical measure.

12. The medical device system of claim 11, including:
a heart sound sensor configured to sense the cardiac mechanical information from the patient and a cardiac electrical sensor configured to sense the cardiac electrical information from the patient,
wherein the signal receiver circuit is configured to receive the sensed cardiac mechanical information from the heart sound sensor and the cardiac electrical information from the cardiac electrical sensor.

13. The medical device system of claim 1, wherein to control the mode or operation of the medical device system includes to control sensing, storage, or transmission of the cardiac electrical information or the cardiac mechanical information or display of detected arrhythmia episodes to optimize resources of the medical device system.

14. The medical device system of claim 1, wherein the assessment circuit is configured to:
perform frequency analysis on an ensemble average of cardiac mechanical information over the detected arrhythmia episode to determine a value of frequency density of the ensemble average of cardiac mechanical information, the ensemble average representative of an aggregate of cardiac mechanical information over multiple cardiac cycles of the detected arrhythmia episode; and
determine the type of the detected arrhythmia episode based on the determined value of frequency density of the ensemble average of cardiac mechanical information.

15. A method for improving arrhythmia detection by a medical device system to optimize resources of the medical device system, comprising:
receiving physiologic information of a patient using a signal receiver circuit, the physiologic information including cardiac electrical and cardiac mechanical information; and
using an assessment circuit:
detecting an arrhythmia episode using the cardiac electrical information;
analyzing the cardiac mechanical information occurring over a final portion of a cardiac cycle of the detected arrhythmia episode to confirm the detected arrhythmia episode or to determine a type of the detected arrhythmia episode based on analysis of the cardiac mechanical information; and
providing a control signal to control a mode or operation of the medical device system based on the confirmation of the detected arrhythmia episode or the determined type of the detected arrhythmia episode to optimize resources of the medical device system.

16. The method of claim 15, comprising:
triggering sensing of or receiving the cardiac mechanical information, including heart sound information, based on the detected arrhythmia episode.

17. The method of claim 15, wherein receiving the cardiac electrical information includes receiving heart rate information,
wherein analyzing the cardiac mechanical information includes confirming the detected arrhythmia episode and to determine the type of the detected arrhythmia episode based on the analysis of the cardiac mechanical information,
wherein determining the type of arrhythmia episode includes determining one of a plurality of types of arrhythmia episode including an atrial tachycardia episode, an atrial fibrillation episode, or an atrial flutter episode.

18. The method of claim 17, comprising:
triggering sampling and storing an electrocardiogram signal at a sampling rate and storage size greater than the heart rate information in response to the cardiac mechanical information exceeding a threshold.

19. The method of claim 15, wherein receiving the cardiac mechanical information includes receiving an S4 heart sound value,
wherein analyzing the cardiac mechanical information includes determining and analyzing the S4 heart sound value occurring over an S4 window of a first cardiac cycle and ending at a beginning of a second cardiac cycle immediately following the first cardiac cycle at a subsequent R wave or S1 heart sound.

20. The method of claim 19, wherein analyzing the S4 heart sound value comprises detecting a change in the S4 heart sound value for the detected arrhythmia episode with respect to a baseline value and confirming the detected arrhythmia episode and determining the type of the detected arrhythmia episode based on the detected change.
